(19) United States
(12) Patent Application Publication
(10) Pub. No.: US 2010/0240966 A1
(43) Pub. Date: Sep. 23, 2010

(54) LIFESTYLE HABIT IMPROVEMENT SUPPORTING APPARATUS AND METHOD THEREOF

(76) Inventors: Yuko Taniike, Osaka (JP); Eiji Noguchi, Osaka (JP); Tatsurou Kawamura, Kyoto (JP)

Correspondence Address: WENDEROTH, LIND & PONACK L.L.P., 1030 15th Street, N.W., Suite 400 East, Washington, DC, 20005-1503, US

(21) Appl. No.: 12/160,570

(22) PCT Filed: Jan. 16, 2007
(86) PCT No.: PCT/JP2007/050463

§ 371 Date: Jul. 10, 2008

(30) Foreign Application Priority Data
Jan. 17, 2006 (JP) 2006-009240

(51) Int. Cl. A61B 5/00

(52) U.S. Cl. 600/301

(57) ABSTRACT

A lifestyle habit improvement supporting apparatus which allows a stage, at which the subject working on lifestyle habit improvement is, to be judged more accurately is provided.

The lifestyle habit improvement supporting apparatus which supports lifestyle habit improvement of a subject includes: a measurement value obtaining unit (501) which obtains a measurement value reflecting a lifestyle habit of the subject; and stage judging units (503 to 507) which judge a stage at which the subject working on lifestyle habit improvement is, based on either the measurement value obtained by the measurement value obtaining unit (501), or the number of measurements representing the number of pieces of data of the measurement value obtained by the measurement value obtaining unit (501).

### TECHNICAL FIELD

The present invention relates to a lifestyle habit improvement supporting apparatus and a method thereof for providing an instruction method and advice in lifestyle habit improvement which are suited for a subject required to improve his or her lifestyle.

### BACKGROUND ART

In prevention and treatment of life-related diseases, a basic issue to be dealt with is to know an ideal lifestyle habit suitable for a subject from the subject's current lifestyle, and to improve the ideal lifestyle to a more ideal one. Lifestyle habits include habits such as diet, exercise, smoking and drinking.

Here, the “subject” is a target of lifestyle habit improvement, and is required to improve a lifestyle habit due to his or her lifestyle disease or a possible lifestyle disease, which is applied to the description below.

The stages change model, a theory of behavioral science, has conventionally been adopted for an effective instruction in lifestyle habit improvement. Specifically, the stages change model is an instruction method which comprehends a state of object people to treatment (knowledge, willingness, and a practice degree) to classify the object people into stages, and practices an instruction based on each of the stages. Such a behavioral science theory enables an instruction based on a state of the subjects. First, a stage of subjects needs to be determined in order to adopt the stages change model into treatment.

A questionnaire has been used as a conventional determination scheme of a stage of the subjects. In other words, a health care provider asks the subject questions regarding a degree of motivation for lifestyle improvement, and classifies the stage of the subjects into five stages; namely, the “precontemplation stage”, “contemplation stage”, “preparation stage”, “action stage”, and “maintenance stage” based on the responses.

As an example of a state regarding improvement of living in each of the stages, the “precontemplation stage” indicates a state with no willingness to improvement of life The “contemplation stage” indicates a state in which there is willingness towards lifestyle improvement although not yet properly set into action. The “preparation stage” indicates a state that the subject is working on or used to work on lifestyle improvement in his or her way. The “action stage” indicates a state that the subject has been working on lifestyle improvement in a correct manner for not more than six months. The “maintenance stage” indicates a state that the subject has been working on the lifestyle improvement for more than six months.

An example of a conventional stage determination scheme applied to giving up smoking shall be described, referring to FIGS. 1 and 2.

FIG. 1 shows an example of a question and responses in the conventional stage determination scheme. To a question “Do you want to give up smoking?”, the example shown in the drawing assumes five responses; that is, “5) No, I will not start giving up smoking for the next six months”, “4) No, but I think I will start giving up smoking within six months”, “3) No, but I think I will start giving up smoking within a month (I used to give up smoking)”, “2) Yes, I have given up smoking for less than six months”, and “1) Yes, I have given up smoking for six months or more”.

FIG. 2 shows a correspondence between a response result and an associated stage according to the conventional stage determination scheme. The drawing shows a correspondence between each of the responses to the question shown in FIG. 1 and an associated stage.

To the question “Do you want to give up smoking?”, the drawing exemplifies a stage of the subject responding: “5) No, I will not start giving up smoking for the next six months” as the “precontemplation stage”; “4) No, but I think I will start giving up smoking within six months” as the “contemplation stage”; “3) No, but I think I will start giving up smoking within a month (I used to quit smoking)” as the “preparation stage”; “2) Yes, I have given up smoking for less than six months” as the “action stage”; and “1) Yes, I have given up smoking for six months or more” as the “maintenance stage” (See Non-patent Reference 1, for example).

Lifestyle habit improvement supporting apparatuses utilizing the stages change model have conventionally been proposed, as well (See Patent Reference 1 and Patent Reference 2, for example).

The apparatus described in Patent Reference 1 displays a question for checking out willingness to lifestyle habit improvement, and a user inputs a response to the question. The apparatus is configured to assign, based on the response, a stage showing the user's phased willingness to working on.

Moreover, on the apparatus in Patent Reference 2, a healthcare instructor conducts an interview with a person subject to healthcare instruction, watching a monitor displaying a question for confirming the person subject to healthcare instruction's awareness level of lifestyle habit improvement, then the healthcare instructor inputs a response from the person subject to healthcare instruction. Based on the response, determination is made to which stage the awareness level of the person subject to healthcare instruction belongs.

[Non-patent Reference 1] Hata Ei-ichi., and Yuriko Doi, “Ko-do Kagaku—Kenkozukuri no Tame no Riron to o-yo (Theory and Application for Health Promotion)”, Nan-ei Do, Oct. 25, 2003, pp. 73-75.

### [Patent Reference 1] Japanese Unexamined Patent Application Publication No. 2001-22837

### [Patent Reference 2] Japanese Unexamined Patent Application Publication No. 2002-358372

### DISCLOSURE OF INVENTION

### Problems that Invention is to Solve

Conventional techniques comprehend a lifestyle habit of a subject, using a questionnaire to the subject in order to determine a stage of the subject. This causes a problem in that the stage of the subject cannot be necessarily determined with accuracy.

In the case where an instruction in salt reduction is provided, for example, a thorough interview survey of the subject's diet needs to be conducted in order to accurately comprehend the state of the subject in salt reduction.

Since an interview is based on memory of the subject or details of the diet which the subject records, the interview survey can be inaccurate. If the response from the subject regarding the details of the diet is inaccurate, an accurate understanding of the subject's state will be difficult. As a result, the stage of the subject cannot be correctly determined.

Furthermore, in the case where the question has a clear purpose as mentioned above, the subject senses an intention of the question and may provide a response which does not conform with the state of the subject. If the subject provides the response which does not conform with the state of the subject, the state of the subject cannot be accurately comprehended. As a result, the stage of the subject cannot be correctly determined. Thus, there have been some cases where a stage of a subject cannot be correctly determined.

In addition, such a questionnaire is so troublesome that the subject often makes a mistake, such as forgetting recording the details of the diet, which results in an inaccurate response from the subject.

In view of the above problems, the present invention has an object of providing a lifestyle habit improvement supporting apparatus and a method thereof which enable a more accurate determination of a stage reflecting a phase in which the subject working on his or her lifestyle habit.

### Means to Solve the Problems

In order to achieve the above objectives, the lifestyle habit improvement supporting apparatus in the present invention supports lifestyle habit improvement of a subject, and includes: a measurement value obtaining unit which obtains a measurement value reflecting a lifestyle habit of the subject; and a stage judging unit which judges a stage at which the subject working on lifestyle habit improvement is, based on either the measurement value obtained by the measurement value obtaining unit, or the number of measurements representing the number of pieces of data of the measurement value obtained by the measurement value obtaining unit.

### EFFECTS OF THE INVENTION

According to a lifestyle habit improvement supporting apparatus and a method thereof in the present invention, a stage in which a subject is can be correctly determined, and a more appropriate instruction and advice can be provided to the subject.

### BRIEF DESCRIPTION OF DRAWINGS

FIG. 1 shows an example of a question and answers in a conventional stage determination scheme.

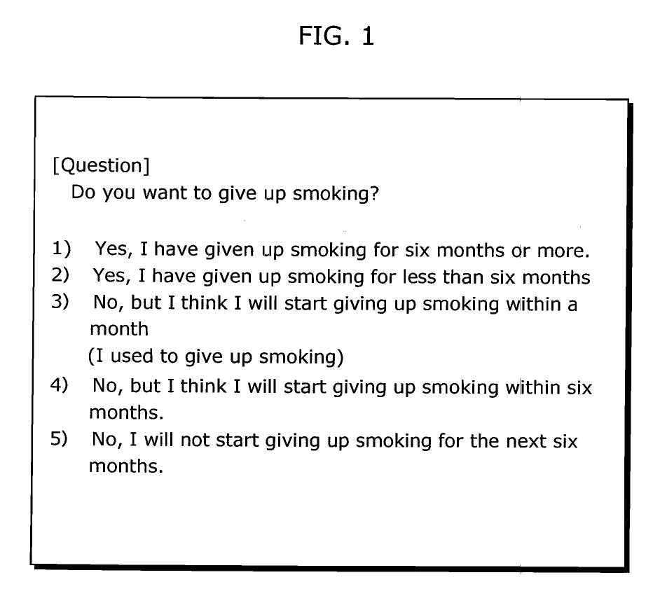

FIG. 2 shows a correspondence between a response result and an associated stage according to the conventional stage determination scheme.

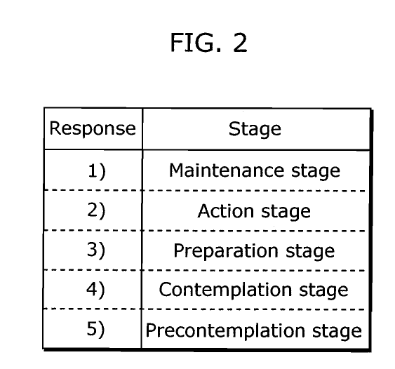

FIG. 3 is a schematic view showing a configuration of a lifestyle habit improvement supporting system in a first embodiment.

FIG. 4 is a block diagram showing a functional structure of a urine component measuring instrument and the lifestyle habit improvement supporting apparatus in the lifestyle habit improvement supporting system of the first embodiment.

FIG. 5 is a diagram showing an example of a stage instruction content table which is stored in an instruction content storing unit in the first embodiment.

FIG. 6 is a diagram showing an example of information in a lifestyle habit improvement stored in a recording medium in the first embodiment.

FIG. 7 is a block diagram showing a structure of a function performed by a CPU executing a first program stored in a determination program storing unit 408 in the first embodiment.

FIG. 8 is a diagram showing a first example of threshold value data stored in a threshold value storing unit.

FIG. 9 is a flowchart of processing executed on the urine component measuring instrument in the first embodiment.

FIG. 10 is a drawing showing an input request example, of a probable value, displayed on a display unit of the urine component measuring instrument in the first embodiment.

FIG. 11 is a drawing showing an example of a measuring chip attachment request displayed on the display unit of the urine component measuring instrument in the first embodiment.

FIG. 12 is a drawing showing an example of a message notifying that measurement preparation, displayed on the display unit of the urine component measuring instrument in the first embodiment, is ready.

FIG. 13 is a drawing showing an example of a message notifying that measurement preparation, displayed on the display unit of the urine component measuring instrument in the first embodiment, is ready.

FIG. 14 is a drawing showing an example of the probable value, a target value, and the measurement value displayed on the display unit of the urine component measuring instrument in the first embodiment.

FIG. 15 is a flowchart showing a flow of processing executed on a lifestyle habit improvement supporting apparatus 4 in the first embodiment.

FIG. 16 is a display example of a message, prompting an insert of the storing medium, displayed on the lifestyle habit improvement supporting apparatus in the first embodiment.

FIG. 17 is a drawing showing a display example, of a stage at which a subject is and an instruction content, displayed on the lifestyle habit improvement supporting apparatus in the first embodiment.

FIG. 18 is a flowchart showing a first example of details of processing which the CPU executes in stage and instruction content determining processing S204 shown in FIG. 15.

FIG. 19 is a schematic view showing a first modification example of the configuration of the lifestyle habit improvement supporting system in the first embodiment.

FIG. 20 is a block diagram showing a structure of a function performed by the CPU executing a second program stored in the determination program storing unit 408 in a second modification of the first embodiment.

FIG. 21 is a diagram showing a second example of threshold value data stored in a threshold value storing unit 502.

FIG. 22 is a flowchart showing a second example of details of processing which the CPU executes in stage and instruction content determining processing S204 shown in FIG. 15.

FIG. 23 is a block diagram showing a functional structure on a lifestyle habit improvement supporting apparatus in a second embodiment.

FIG. 24 is a flowchart showing processing executed on a lifestyle habit improvement supporting apparatus 10 in the second embodiment.

FIG. 25 is a drawing showing a display example, of a stage at which a subject is, displayed on a lifestyle habit improvement supporting apparatus in the second embodiment.

### NUMERICAL REFERENCES

1 Urine component measuring instrument
2 Storing medium
3 Measuring chip
4 and 10 Lifestyle habit improvement supporting apparatus
101 and 406 Power button
102 and 401 Input unit
103 and 403 Displaying unit
104 and 402 Storing medium insertion slot
105 Measuring chip connecting port
106 and 404 CPU
107 and 405 Power source
108 and 407 Timer
109 Attachment detecting unit
110 Terminal for measuring chip
111 Voltage applying unit
112 Electric signal measuring unit
113 and 410 Terminal for storing medium
114 and 408 Determination program storing unit
115 and 409 Instruction content storing unit
116 Stage determination button
301 Electrode

### BEST MODE FOR CARRYING OUT THE INVENTION

The lifestyle habit improvement supporting apparatus in the present invention supports lifestyle habit improvement of a subject, and includes: a measurement value obtaining unit which obtains a measurement value reflecting a lifestyle habit of the subject; and a stage judging unit which judges a stage at which the subject working on lifestyle habit improvement is, based on either the measurement value obtained by the measurement value obtaining unit, or the number of measurements representing the number of pieces of data of the measurement value obtained by the measurement value obtaining unit.

This allows a more appropriate instruction and advice to be provided to the subject since a stage, at which the subject working on lifestyle habit is, is judged according to subjective data; namely the measurement value measured by a measurement device, so that the stage of the subject can be accurately judged.

Preferably, the lifestyle habit improvement supporting apparatus further includes a target number of measurements obtaining unit which obtains the target number of measurements showing the target number of times for which the subject measures the measurement value, wherein the stage judging unit judges the stage at which the subject working on lifestyle habit improvement is, based on the number of measurements, and the target number of measurements.

Further, the lifestyle habit improvement supporting apparatus further includes a target measurement value obtaining unit which obtains a target measurement value showing a target of the measurement value of the subject, wherein the stage judging unit judges the stage at which the subject working on lifestyle habit improvement is, based on the measurement value, the number of measurements, and the target measurement value.

As described above, use of the target number of measurements and the target measurement value for judging the stage of the subject enables the stage of the subject to be classified in more detail, so that the stage can be judged with more accuracy.

Preferably, the measurement value obtaining unit obtains the measurement value corresponding to a measurement date within a certain period, and the stage judging unit judges the stage at which the subject working on lifestyle habit improvement is, based on either the measurement value or the number of measurements corresponding to the measurement date within the certain period.

As described above, the stage, at which the subject working on lifestyle habit improvement is, is determined based on appropriately selected data for a period, so that a more appropriate stage judgment can be made.

In addition, preferably, the measurement value obtaining unit obtains the measurement value and a probable value which is a measurement value that the subject assumes, and the stage judging unit judges the stage at which the subject working on lifestyle habit improvement is, based on the measurement value, the probable value, and the number of measurements.

The measurement value obtaining unit obtains the measurement value and the probable value which correspond to a measurement date within a certain period, and the stage judging unit judges the stages at which the subject working on lifestyle habit improvement is, based on the measurement value, the number of measurements, and the probable value which correspond to the measurement date within the certain period.

In the case where the subject has correct knowledge on lifestyle habit improvement, the probable value and the measurement value become close each other. Hence, the stage judgment, utilizing the probable value and the measurement value which the subject inputs, allows the stage, at which the subject working on lifestyle habit improvement is, to be judged, considering, as well, whether or not the subject has correct knowledge on lifestyle habit improvement.

Further, preferably, the lifestyle habit improvement supporting apparatus includes an instruction deciding unit which decides an instruction method and advice suitable for the stage at which the subject working on lifestyle habit improvement is, by referring, in accordance with a result of the judgment by the stage judging unit, to a table storing stages of working on lifestyle habit improvement, and instruction methods and advice, suitable for the stage of working for lifestyle habit improvement is.

As mentioned above, on the lifestyle improvement supporting apparatus, the subject himself or herself judging the stage at which the subject working on lifestyle habit improvement is and an instructor for lifestyle habit improvement to the subject can refer to an instruction method and advice, based on an accurate stage judgment, provided by the lifestyle habit improvement supporting apparatus. Further, the subject can work on his or her lifestyle habit improvement with appropriate advice obtained, so that the lifestyle habit improvement can be effectively worked on.

In addition, preferably, the stage judging unit includes a first stage judging sub-unit which compares the number of measurements and a first threshold value, and, based on a result of the comparison, judges the stage at which the subject working on lifestyle habit improvement is as a first stage.

Moreover, the first stage judging sub-unit compares the number of measurements and the first threshold value, and, in the case where the number of measurements is below the first threshold value, judges the stage at which the subject working on lifestyle habit improvement is as a precontemplation stage in which the subject is uninterested in lifestyle habit improvement.

As described above, whether or not the subject is at the first stage in lifestyle habit improvement based on objective data; for example, whether or not the subject is interested in lifestyle habit improvement, can be judged, by judging the stage at which the subject working on lifestyle habit improvement is, utilizing a total number of pieces of the data of the measurement value.

In addition, preferably, the stage judging unit includes a second stage judging sub-unit compares the number obtained by multiplying the target number of measurements by a second threshold value and the number of measurements, and, based on a result of the comparison, judges the stage at which the subject working on lifestyle habit improvement is as a second stage.

Further, the second stage judging sub-unit compares the number obtained by multiplying the target number of measurements by the second threshold value and the number of measurements, and, in the case where the number is equal to or less than the number of measurements, to judge the stage at which the subject working on lifestyle habit improvement is as a contemplation stage in which the subject starts to be interested in lifestyle habit improvement.

As described above, whether or not the subject is at the second stage in lifestyle habit improvement based on objective data; for example, whether or not the subject starts to get interested in lifestyle habit improvement, can be judged, by judging the stage at which the subject working on lifestyle habit improvement is, utilizing a total number of pieces of the data of the measurement value.

Further, preferably, the stage judging unit includes a third stage judging sub-unit which compares a ratio of the number of pieces of data to the number of measurements and a third threshold value, and, judges, based on a result of the comparison, the stage at which the subject working on lifestyle habit improvement is as a third stage, the data having a discrepancy degree between the measurement value and the probable value to be equal to or less than a certain value.

Moreover, the third stage judging sub-unit compares the ratio of the number of pieces of data to the number of measurements and the third threshold value, and, in the case where the ratio is smaller than the third threshold value, judges the stage at which the subject working on lifestyle habit improvement is as a first preparation stage in which the subject starts working on lifestyle habit improvement but has not acquired correct knowledge yet, the data having the discrepancy degree between the measurement value and the probable value to be equal to or less than a certain value.

As described above, whether or not the subject is at the third stage based on objective data; for example, whether or not the subject has acquired correct knowledge on lifestyle habit, can be judged, by judging the stage at which the subject working on lifestyle habit improvement is, utilizing the measurement value and the probable value.

In addition, preferably, the stage judging unit includes a fourth stage judging sub-unit which compares a ratio of the number of pieces of data to the number of measurements and a fourth threshold value, and, based on a result of the comparison, judges the stage, at which the subject working on lifestyle habit improvement is, to be a fourth stage, the data indicating that the measurement value is equal to or less than the target measurement value.

Furthermore, the fourth stage judging sub-unit compares the ratio of the number of pieces of data to the number of measurements and the fourth threshold value, and, in the case where the ratio is smaller than the fourth threshold value, judges the stage at which the subject working on lifestyle habit improvement is as a second preparation stage in which the subject is short of utilizing acquired correct knowledge, the data indicating that the measurement value is equal to or less than the target measurement value.

Moreover, the fourth stage judging sub-unit compares the ratio of the number of pieces of data to the number of measurements and the fourth threshold value, and, in the case where the ratio is smaller than the fourth threshold value, judges the stage at which the subject working on lifestyle habit improvement is as a preparation stage in which the subject starts working on lifestyle habit improvement, the data indicating that the measurement value is equal to or less than the target measurement value.

As described above, whether or not the subject is at the fourth stage in lifestyle habit improvement based on objective data; for example, whether or not the subject: utilizes the acquired correct knowledge for lifestyle habit improvement; or has started working on his or her lifestyle habit improvement, utilizing the number of pieces of the data and the target number of measurements of the measurement value.

In addition, preferably, the stage judging unit includes a fifth stage judging sub-unit which compares a length of a period and a fifth threshold value, and, based on a result of the comparison, judges the stage at which the subject working on lifestyle habit improvement is as either a fifth stage or a sixth stage, the length of the period representing a period that the ratio of the number of pieces of data to the number of measurements is equal to or more than the fourth threshold value, and the data indicating that the measurement value is equal to or less than the target measurement value.

Moreover, the fifth stage judging sub-unit compares the length of the period and the fifth threshold value, and, in the case where the length of the period is shorter than the fifth threshold value, judges the stage at which the subject working on lifestyle habit improvement is as an action stage in which the subject begins working on lifestyle habit improvement, utilizing correct knowledge, the length of the period representing the period that the ratio of the number of pieces of data to the number of measurements is equal to or more than the fourth threshold value, the data indicating that the measurement value is equal to or less than the target measurement value.

Further, the fifth stage judging sub-unit compares the length of the period and the fifth threshold value, and, in the case where the length of the period is equal to or more than the fifth threshold value, judges the stage at which the subject working on lifestyle habit improvement is as a maintenance stage in which an appropriate lifestyle habit has been established for health of the subject, the length of the period representing the period that the ratio of the number of pieces of data to the number of measurements is equal to or more than the fourth threshold value, the data indicating that the measurement value is equal to or less than the target measurement value.

As described above, that the subject is at either the fifth stage or the sixth stage in lifestyle habit improvement based on objective data can be judged, by judging the stage at which the subject working on lifestyle habit improvement is, utilizing the measurement value and the probable value. At the fifth stage, the subject has started to work on lifestyle habit improvement, utilizing the acquired correct knowledge, for example, and at the sixth stage, an appropriate lifestyle habit for the health of the subject has established.

In addition, preferably, the value reflecting the lifestyle habit of the subject represents either salinity concentration, or an amount of salt in urine of the subject.

The salinity concentration in urine is one of the values reflecting the lifestyle habit of the subject since salt intake of the subject can be calculated out of a measurement value of the salinity concentration in urine. Thus, lifestyle habit improvement regarding the salt intake of the subject can be supported in terms of the measurement value of the salinity concentration in urine.

Moreover, preferably, the lifestyle habit improvement supporting apparatus further includes a first displaying controlling unit which causes, to be displayed, at least one of: the result of the judgment by the stage judging unit; or one of the instruction method or the advice decided by the instruction deciding unit.

In addition, the lifestyle habit improvement supporting apparatus further includes a second displaying controlling unit which causes the measurement value, the probable value, the target number of measurements, and the target measurement value to be displayed for a certain number of measurements.

As described above, the judgment result, the advice and instruction method, and various values can be displayed with the first displaying controlling unit included in the lifestyle habit improvement supporting apparatus. In addition, with the second displaying controlling unit included in the lifestyle habit improvement supporting apparatus, the subject understands a situation in which the subject working on lifestyle habit improvement is out of the content displayed by the displaying unit, so that the subject can be motivated to work on lifestyle habit improvement. Furthermore, the instructor for lifestyle habit improvement to the subject can provide an instruction of lifestyle habit improvement out of an accurate judgment result.

Further, preferably, the lifestyle habit improvement supporting apparatus further includes: a measuring unit which measures the measurement value; a target receiving unit which receives an input of a target measurement value showing a target of the measurement value of the subject, and the target number of measurements showing the target of the number of times for which the subject measures the measurement value; and a storing unit which stores a measurement date and time when the subject measures, the target measurement value and the target number of measurements received by the target receiving unit, and the measurement value measured by the measuring unit, wherein the measurement value obtaining unit obtains, from the storing unit, the measurement date and time, the target measurement value, the target number of measurements, and the measurement value.

In addition, the lifestyle habit improvement supporting apparatus further includes: a measuring unit which measures the measurement value; a target receiving unit which receives an input of a target measurement value showing a target of the measurement value of the subject, and the target number of measurements showing the target of the number of times for which the subject measures the measurement value; a probable value receiving unit which receives an input of a probable value; and a storing unit which stores a measurement date and time when the subject measures, the target measurement value and the target number of measurements received by the target receiving unit, the measurement value measured by the measuring unit, and the probable value received by the probable value receiving unit, wherein the measurement value obtaining unit obtains, from the storing unit, the measurement date and time, the target measurement value, the target number of measurements, the measurement value, and the probable value.

As described above, with a measuring instrument and an input device included in the lifestyle habit improvement supporting apparatus, the subject himself or herself can judge the stage, so that the subject has increasing opportunities to pay attention to lifestyle habit improvement, and can be motivated to work on lifestyle habit improvement.

Moreover, preferably, the measurement value obtaining unit obtains the measurement value, outside the lifestyle habit improvement supporting apparatus, from a storing unit which is a dismountable storing medium.

This allows a client to facilitate to carry data stored on the storing unit, which improves convenience.

It is noted that the present invention can be implemented not only as the lifestyle habit improvement supporting apparatus including the units mentioned above, but also as: a lifestyle habit improvement supporting method having the units included in the lifestyle habit improvement supporting apparatus as steps; and as a program which causes a computer to execute the units included in the lifestyle habit improvement supporting apparatus as steps. Thus, such a program can be distributed via a recording medium such as a CD-ROM (Compact Disc-Read Only Memory), and a communication network such as the Internet. Moreover, the present invention can be implemented as a lifestyle habit improvement supporting system including those units.

Following is a detailed description of embodiments of the present invention, referring to the drawings.

### First Embodiment

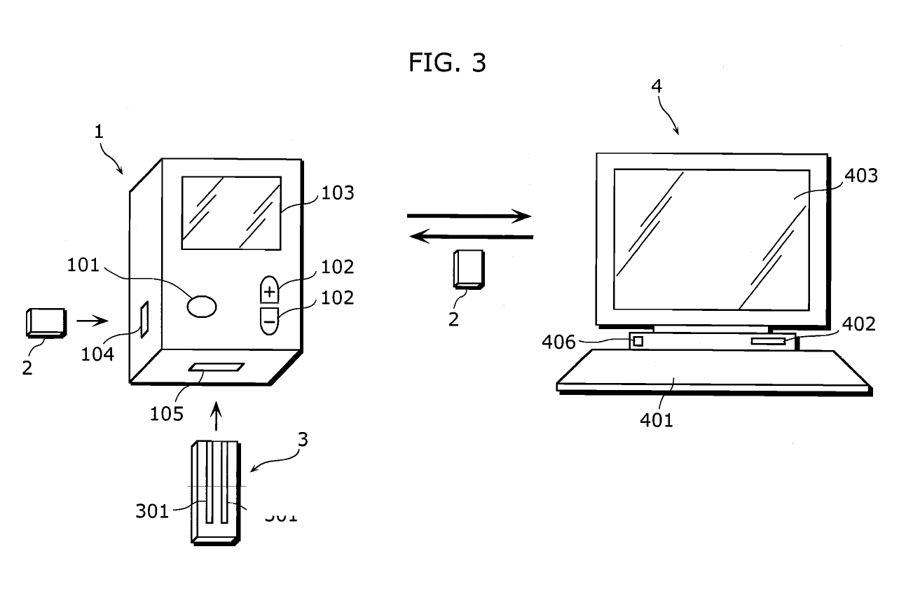

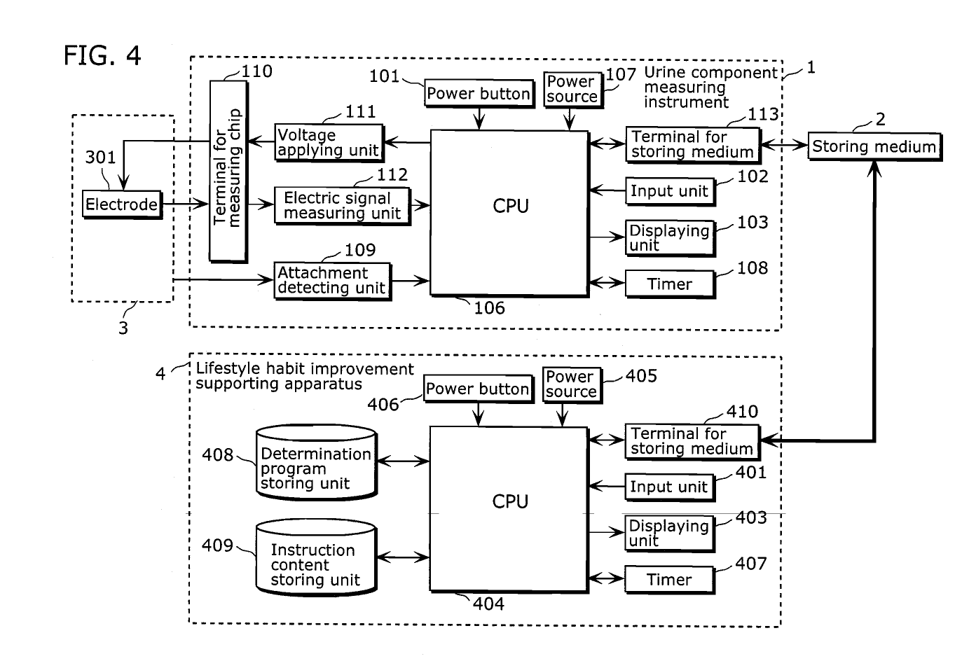

First, a configuration of a lifestyle improvement supporting system in a first embodiment shall be described, using FIGS. 3 and 4.

FIG. 3 is a schematic view showing the configuration of the lifestyle improvement supporting system in the first embodiment. In the lifestyle habit improvement supporting system in the first embodiment, by exchanging information between a urine component measuring instrument and a lifestyle habit improvement supporting apparatus via a storing medium, the lifestyle habit improvement supporting apparatus determines a stage on which a subject working on lifestyle habit improvement is from a measurement value of salinity concentration in urine, of the subject, measured using an urine component measuring instrument. The lifestyle habit improvement supporting system includes a urine component measuring instrument 1 and the lifestyle habit improvement supporting apparatus 4.

Here, the “stage” is based on the stages change model of behavioral science. Stages in the embodiment, basically classified into five stages, include: “precontemplation stage”; “contemplation stage”; “preparation stage”; “action stage”; and “maintenance stage”, and the “preparation stage” is further classified into two stages; namely, “a first preparation stage” and “a second preparation stage”.

The urine component measuring instrument 1 in the first embodiment measures salinity concentration in urine, and includes a power button 101, an input unit 102, a displaying unit 103, a storing medium insertion slot 104, and a measuring chip connecting port 105.

With the subject pressing, the power button 101 switches between power-on and power-off on the urine component measuring instrument 1.

The input unit 102 is an operation processing unit which a user utilizes when inputting information, and includes a numerical key, a keyboard, a touch panel and a voice input instrument, for example.

The displaying unit 103 displays the measurement value measured by the urine component measuring instrument 1 and the information inputted via the input unit 102, and includes an LCD (Liquid Crystal Display), for example.

The storing medium insertion slot 104 is an opening into which the storing medium 2 is inserted. The storing medium, including a memory card, for example, 2 stores information in lifestyle habit improvement, such as the amount of salt in urine which the subject measures.

The measuring chip connecting port 105 is an opening for connecting the measurement chip 3 on which a pair of electrodes 301 is placed. The subject connects the measurement chip 3 with the urine adhered to the measuring chip connecting port 105, and measures the amount of salt in the urine.

Here, the measurement chip 3 has an insulative substrate, made from resin, on which the pair of electrodes 301 is placed. The measuring chip 3 may be either disposable or reused by washing after use.

The lifestyle habit improvement supporting apparatus 4 also includes an input unit 401, a storing medium insertion slot 402 and a displaying unit 403. The lifestyle habit improvement supporting apparatus 4 reads out the measurement value from the storing medium 2 inserted into the storing medium insertion slot 402, and then determines a stage of the subject based on the measurement value, and includes a PC (Personal Computer), for example.

The input unit 401 receives an input into the lifestyle habit improvement supporting apparatus 4 by the user, and includes a keyboard and a mouse, for example.

The storing medium insertion slot 402 is an opening into which the storing medium 2 is inserted, and has an identical function to the storing medium insertion slot 104 included in the urine component measuring instrument 1.

The displaying unit 403, including a monitor, for example, displays the subject's input which the input unit 401 receives, a content read out from a storing medium, and a result of processing in the lifestyle habit improvement supporting apparatus 4.

FIG. 4 is a block diagram showing a functional structure of the urine component measuring instrument 1 and the lifestyle improvement supporting system 4 in the lifestyle habit improvement supporting system of the first embodiment.

The urine component measuring instrument 1 includes the power button 101, the input unit 102, the displaying unit 103, a CPU (Central Processing Unit) 106, a power source 107, a timer 108, an attachment detecting unit 109, a terminal for measuring chip 110, a voltage applying unit 111, an electric signal measuring unit 112, and a terminal for storing medium 113. The descriptions for the power button 101, the input unit 102, and the displaying unit 103 have been provided, referring to FIG. 3, and shall be omitted here.

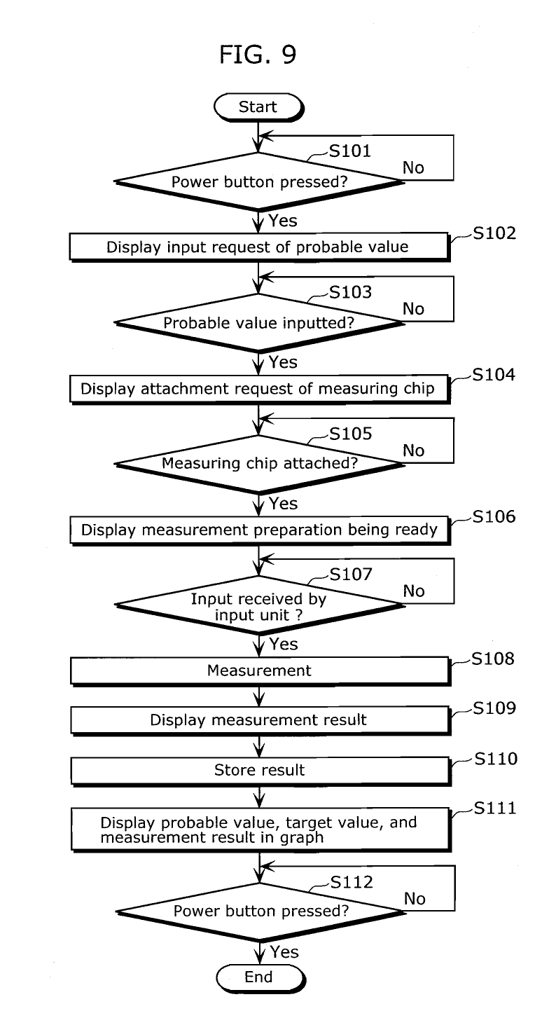

The CPU 106 executes a various kinds of processing. Details which the CPU 106 executes shall be described along with related units included in the urine component measuring instrument 1, as well as referring to FIG. 9.

When the power button 101 is pressed and the power is on, the power source 107 supplies electricity to the CPU 106.

The timer 108, a clock, for example, measures time. Upon receiving an obtainment request of a time outputted from the CPU 106, the timer 108 outputs the time to the CPU 106.

The attachment detecting unit 109 detects the fact that the measurement chip 3 is connected to the measuring chip connecting port 105. Upon detecting the fact that the measuring chip 3 is connected to the measuring chip connecting port 105, the attachment detecting unit 109 notifies the CPU 106 of the fact.

The terminal for measuring chip 110, a metal terminal, for example, electrically connects to the pair of electrodes 301 on the measurement chip 3.

The voltage applying unit 111, according to control by the CPU 106, applies a voltage to the pair of electrodes 301 on the measurement chip 3 via the terminal for measuring chip 110.

The electric signal measuring unit 112 measures an electric signal obtained when the voltage applying unit 111 applies the voltage between the pair of electrodes 301 on the measurement chip 3 via the terminal for measuring chip 110. Here, the “electric signal” is either a value of resistance or conductivity, for example. In other words, the electric signal measuring unit 112 measures a value of resistance or conductivity between the pair of electrodes 301 on the measurement chip 3 when the voltage applying unit 111 applies the voltage.

Salinity concentration in urine is measured by converting the resistance or the conductivity measured by the electric signal measuring unit 112 into salinity concentration, based on the voltage which the voltage applying unit 111 applies from the terminal for measuring chip 110 to the pair of the electrodes 301 on the measurement chip 3 with the subject's urine adhered.

The terminal for storing medium 113, a memory card reader shown as an example of the storing medium 2, for example, electrically connects the storing medium 2 and the urine component measuring instrument 1.

Using the urine component measuring instrument 1 having the above functions, the subject himself or herself can easily measure salinity concentration in urine. Hereinafter, operations of the urine component measuring instrument 1 are assumed to be performed by the subject. Obviously, a person other than the subject can also perform the operations.

Next, the lifestyle habit improvement supporting apparatus 4 includes the input unit 401, the storing medium insertion slot 402, the displaying unit 403, the displaying unit 403, a CPU 404, a power source 405, a power button 406, a timer 407, a determination program storing unit 408, an instruction content storing unit 409, and a terminal for storing medium 410. The input unit 401, the storing medium insertion slot 402, and the displaying unit 403 have been described in FIG. 3, and the description shall be omitted here.

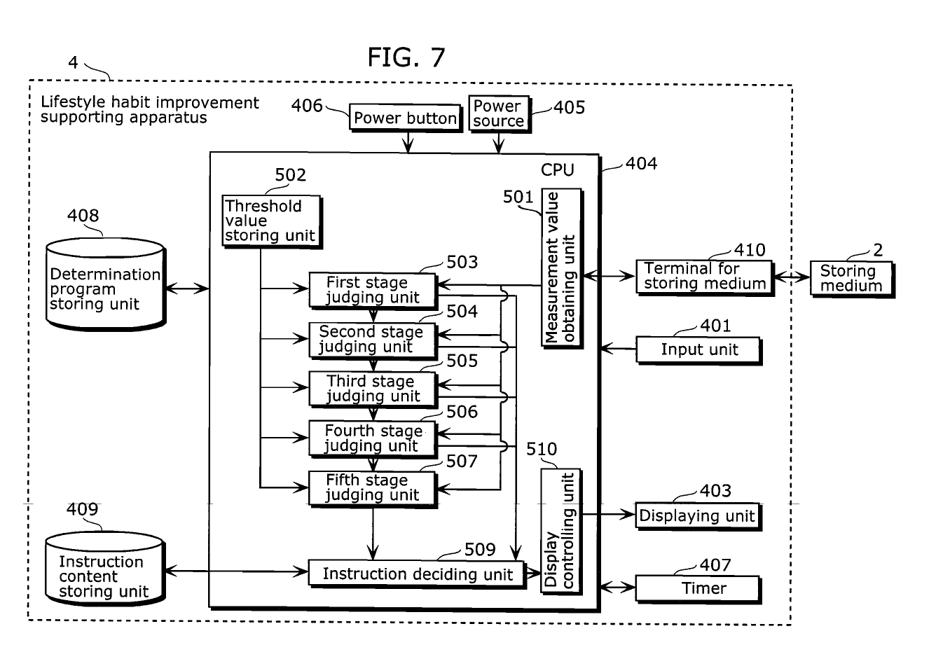

The CPU 404 executes a various kinds of processing. A function and details of processing which the CPU 404 has and executes shall be described along with related units included in the lifestyle habit improvement supporting apparatus 4, as well as referring to FIGS. 7, 13, and 14.

The power source 405 supplies electricity to the CPU 404, and the user pressing, the power button 406 switches between power-on and power-off on the lifestyle habit improvement supporting apparatus 4. When the power button 406 is pressed and the power is on, the power source 405 supplies electricity to the CPU 404.

The timer 407, a clock, for example, measures time. Upon receiving an obtainment request of a time outputted from the CPU 404, the timer 407 outputs the time to the CPU 404.

The determination program storing unit 408, a HDD (Hard Disk Drive), for example, stores a program to determine a stage. The program stored in the determination program storing unit 408 is read out to execute by the CPU 404.

The instruction content storing unit 409, including a HDD (Hard Disk Drive), for example, stores a stage instruction content table which associates a stage with an instruction content.

Here, the “instruction content” is a matter of an instruction provided to a subject in each stage, and includes, for example, a concrete instruction and advice which suit the subject in each stage and an outline of the instruction and advice. When a specialist including a medical doctor uses the lifestyle habit improvement supporting apparatus 4, the advice and the outline of the instruction for each stage may be stored into the instruction content storing unit 409. Referring to the advice and the outline of the instruction given by the lifestyle habit improvement supporting apparatus 4, the expert will be able to provide a concrete instruction and advice which suit the subject.

When a subject himself or herself uses the lifestyle habit improvement supporting apparatus 4; meanwhile, an ordinary subject finds it difficult to apply just a given outline of the instruction and advice to his or her own case. In this case, it is appropriate that a specific instruction and advice for each stage be stored in the instruction content storing unit 409.

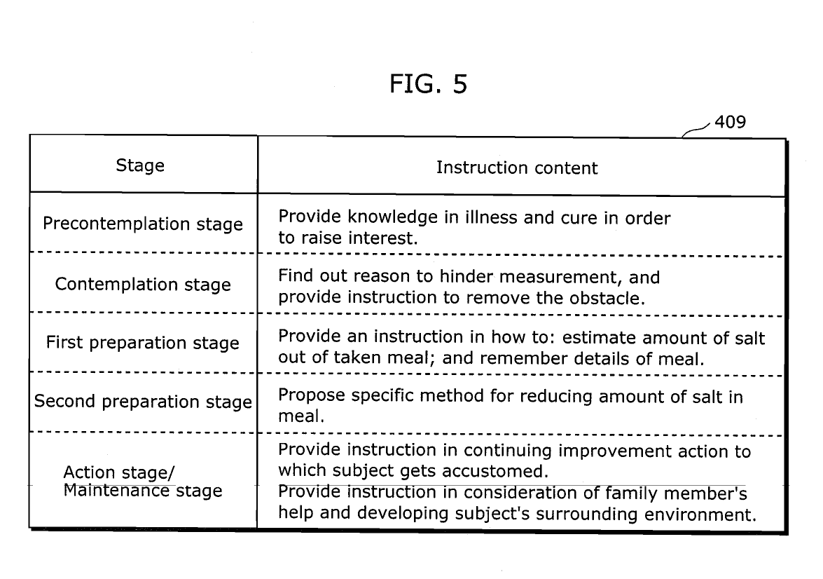

FIG. 5 is a diagram showing an example of a stage instruction content table which is stored in the instruction content storing unit 409 in the first embodiment. The stage instruction content table exemplified in the drawing includes two items; namely, the “stage” and the “instruction content”. The “instruction content” shown in the drawing is not a specific instruction or advice, but an example of an outline of an instruction and advice.

The example shown in the drawing classifies the “stage” into six stages; namely, the “precontemplation stage”, “contemplation stage”, “first preparation stage”, “second preparation stage”, “action stage”, and “maintenance stage”. In the table, an instruction outline associating with each stage is stored in the “instruction content”. Furthermore, in the example in the drawing, the “action stage” and the “maintenance stage” share an identical instruction outline.

The “instruction content” associating with the “precontemplation stage” of the “stage” is “Provide knowledge in an illness and a cure in order to raise the subject's interest”.

Here goes back to the description of FIG. 4.

As well as the terminal for storing medium 113 included in the urine component measuring instrument 1, the terminal for storing medium 410, a memory card reader shown as an example of the storing medium 2, for example, electrically connects the storing medium 2 and the lifestyle habit improvement supporting apparatus 4.

As described above, since the urine component measuring instrument 1 and the lifestyle habit improvement supporting apparatus 4 in the first embodiment include the above functions, the subject can measure salinity concentration in urine by himself or herself to aware improvement of the lifestyle habit. In addition, a measurement date is stored in the storing medium 2, and thus, unlike an interview, a stage at which the subject is can be determined out of accumulated objective data.

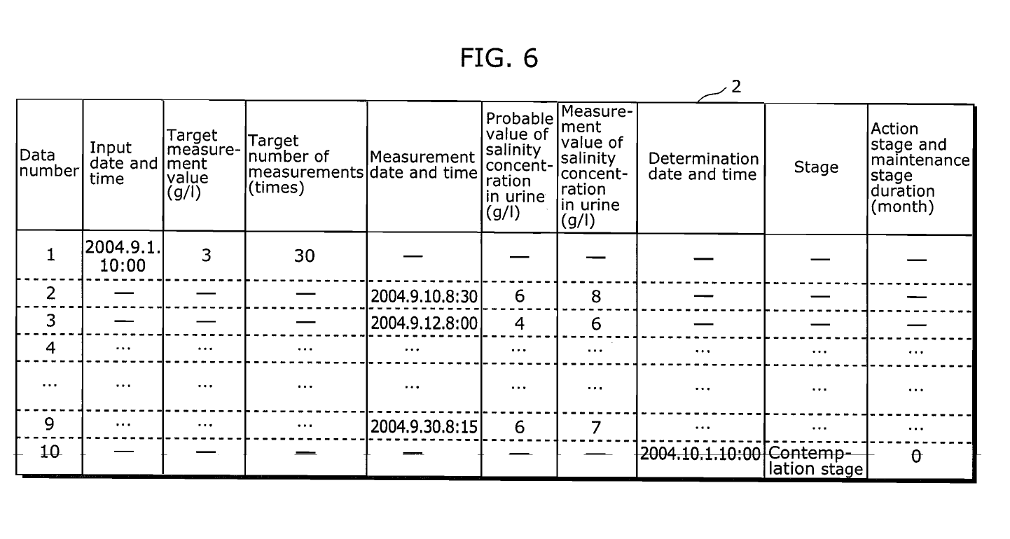

Next, a content stored in the storing medium 2 shall be described, referring to FIG. 6.

FIG. 6 is a diagram showing an example of information in improvement of a lifestyle habit stored in the recording medium 2 in the first embodiment. The information in improvement of a life exemplified in the drawing includes the “data number”, “input date and time”, “target measurement value (g/l)”, “target number of measurements (times)”, “measurement date and time”, “probable value of salinity concentration in urine (g/l)”, “measurement value of salinity concentration in urine (g/l)”, “determination date and time”, “stage”, and “action stage and maintenance stage duration (month)”.

Information to be stored in the “data number” indicates a unique number of the information in improving lifestyle habit stored in the storing medium 2. This information is automatically numbered by the CPU 106 or the CPU 404.

Information to be stored in the “input date and time” indicates a date and time when the “target measurement value (g/l)”, and “target number of measurements (times)” are inputted. The information to be stored in the “input date and time” is: obtained from the timer 407 by the CPU 404 when the “target measurement value (g/l)”, and “target number of measurements (times)” are stored in the storing medium 2; and stored by the CPU 404 along with the “target measurement value (g/l)”, and “target number of measurements (times)”.

Information to be stored in the “target measurement value (g/l)” indicates a target of a measurement value of salinity concentration in urine calculated out of the subject's salt intake which an instructor for lifestyle habit improvement, such as the subject's doctor, sets. Salinity concentration in urine varies according to salt intake. Thus, the subject appropriately controls his or her salt intakes, so that the measurement value of the salinity concentration in urine can be brought near to the target measurement value. The lifestyle habit improvement supporting apparatus 4 stores the information to be stored in the “target measurement value (g/l)”.

Information to be stored in the “target number of measurements (times)” indicates a target number of times which salinity concentration in urine is measured by the subject. The target number of measurements is set by an instructor for lifestyle habit improvement, as the information to be stored in the “target measurement value (g/l) is stored. The information to be stored in the “target number of measurements (times)” is stored by the lifestyle habit improvement supporting apparatus 4.

Information to be stored in the “measurement date and time” indicates a time and date when the measurement value of salinity concentration in urine is measured. The information to be stored in the “measurement date and time” is obtained from the timer 108 by the CPU 106 when the “probable value of salinity concentration in urine (g/l)” and “salinity concentration in urine (g/l)” are stored in the storing medium 2, and stored by the CPU 106 along with the “probable value of salinity concentration in urine (g/l)” and “salinity concentration in urine (g/l)”.

Information to be stored in the “probable value of salinity concentration in urine (g/l)” indicates salinity concentration in urine assumed out of the subject's diet, and is a probable value which the subject inputs with the input unit 102 when the subject measures his or her salinity concentration in urine. The information to be stored in the “probable value of salinity concentration in urine (g/l)” is obtained to store by the CPU 106 via the input unit 102.

Salinity concentration in urine can be calculated out of salt intake from diet. Thus, a subject who acquires correct knowledge in diet can assume a probable value out of the diet.

Information to be stored in the “salinity concentration in urine (g/l)” indicates the measurement value of the subject's salinity concentration in urine measured by the urine component measuring instrument 1. An electric signal inputted from the electric signal measuring unit 112 is converted into salinity concentration in urine by the CPU 106 and stored.

Information to be stored in the “determination date and time” indicates a time and date when a stage at which the subject is is determined. The information to be stored in the “determination date and time” is obtained from the timer 407 by the CPU 404 when the “stage” and “action stage and maintenance stage duration (month)” are stored in a storing medium, and is stored by the CPU 404 along with “stage” and “action stage and maintenance stage duration (month)”.

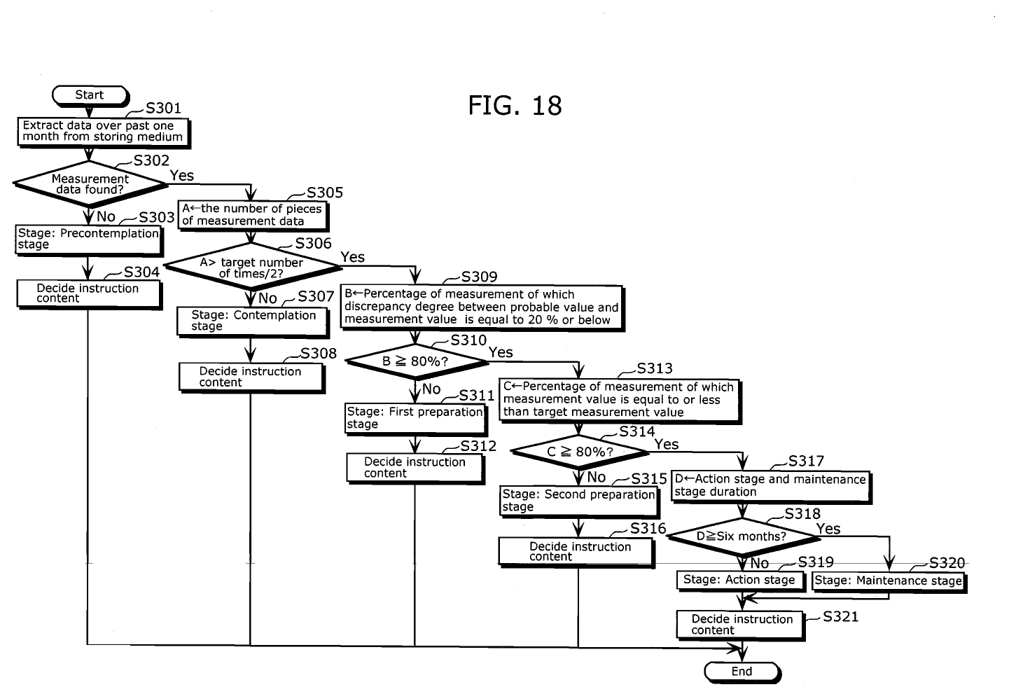

The information to be stored in the “stage”: is a result which the lifestyle habit improvement supporting apparatus 4 determines, using the information stored in the “target measurement value (g/l)”, “target number of measurements (times)”, “probable value of salinity concentration in urine (g/l)”, and “salinity concentration in urine (g/l)”, and indicates a state of the subject working on lifestyle habit improvement. Details of a determination procedure shall be described hereinafter, referring to FIG. 18.

Information to be stored in the “action stage and maintenance stage duration (month)” indicates duration which the subject's “stage” is determined to be either the “action stage” or “maintenance stage”. A specific determination procedure of the information to be stored in the “action stage and maintenance stage duration (month)” shall be described hereinafter, referring to FIG. 18.

Among the above items, the information to be stored in the “target measurement value (g/l)” and “target number of measurements (times)” is inputted on the input unit 401 in the lifestyle habit improvement supporting apparatus 4 by an instructor for lifestyle habit improvement for the subject. These are inputted and stored into the storing medium 2 either when the subject initially uses a health care system in the embodiment of, or a content of a target is changed.

These examples of the information are indicated in “1” in the “data number” exemplified in FIG. 6. The information in lifestyle habit improvement when the “data number” is “1” represents the “target measurement value (g/l)” to be “3” and “target number of measurements (times)” to be “30”. In addition, the “input date and time” when the information is stored is “10:00, Sep. 1, 2004”.

The information to be stored in the “measurement date and time”, “probable value of salinity concentration in urine (g/l)”, and “measurement value of salinity concentration in urine (g/l)” is stored when the subject measuring salinity concentration in urine.

These examples of the information are indicated in “2” through “9” in the “the data number” exemplified in FIG. 6. For example, information shown in “2” in the “data number” represents the “measurement date and time” to be “8:30, Sep. 10, 2004”, the “probable value of salinity concentration in urine (g/l)” to be “6” at the moment of the measurement, and the “salinity concentration in urine (g/l)” to be “8”.

The information to be stored in the “determination date and time”, “stage”, and “action stage and maintenance stage duration (month)” is stored in the lifestyle habit improvement supporting apparatus 4 when measuring a stage.

These examples of the information are indicated in “10” in the “data number” exemplified in FIG. 6. The information in lifestyle habit improvement when the “data number” is “10” represents the “target date and time” to be “10:00, Oct. 1, 2004” and the “stage” determined then to be “contemplation stage”, and the “action stage and maintenance stage duration (month)” to be “0”.

When working with medical information, an association between the information and the subject is important. This measuring apparatus allows each subject to utilize his or her unique storing medium to clarify the association between the result and the subject. As a matter of course, instead of the above scheme, another scheme may be utilized, that is, a unique password may be set for each subject to associated the password with each subject.

FIG. 7 is a block diagram showing a structure of function performed by the CPU 404 executing the first program stored in the determination program storing unit 408 of the first embodiment. The CPU 404 executing the first program stored in the determination program storing unit 408 (referred to as “the first program execution CPU 404, hereinafter): judges a stage for the subject to work on lifestyle improvement according to the measurement value, of the subject's salinity concentration in urine, accumulated in the storing medium 2; and presents an instruction outline based on the judgment.

The first program execution CPU 404 includes a measurement value obtaining unit 501, a threshold value storing unit 502, a first stage judging unit 503, a second stage judging unit 504, a third stage judging unit 505, a fourth stage judging unit 506, a fifth stage judging unit 507, an instruction deciding unit 509, and a display controlling unit 510.

Out of the CPU 401, the lifestyle habit improvement supporting apparatus 4 also has: the input unit 404, the displaying unit 403, the power source 405, the power button 406, the timer 407, the determination program storing unit 408 storing the first program, the instruction content storing unit 409, and the terminal for storing medium 410 which is a connection to the storing medium 2; and exchange information with these. The units which the CPU 404 externally has have been described with reference to FIG. 4, and the description thereof shall be omitted.

The measurement value obtaining unit 501 is an example of a measurement value obtaining unit obtaining a measurement value reflecting the lifestyle habit of the subject. The measurement value obtaining unit 501 is also an example of a measurement value obtaining unit obtaining a measurement value corresponding to a measurement date within a certain period. Further, the measurement value obtaining unit 501 is an example of a unit to obtain a measurement value and a probable value which is a measurement value that the subject assumes. In addition, the measurement value obtaining unit 501 is an example of a unit to obtain a measurement value and a probable value corresponding to a measurement date within a certain period.

Moreover, the measurement value obtaining unit 501 is an example of: the target number of measurements obtaining unit obtaining the target number of measurements showing the target number of times for which the subject measures the measurement value; and a target measurement value obtaining unit obtaining a target measurement value showing a target of a measurement value of the subject.

The measurement value obtaining unit 501 in the embodiment obtains, via the terminal for storing medium 410, the measurement value, the target measurement value, the target number of measurements, and the probable value of the subject stored in the storing medium 2. In the embodiment, the “measurement value” is a measurement value of salinity concentration in urine, and the “probable value” is a measurement value of salinity concentration in urine which the subject assumes.

The threshold value storing unit 502 stores threshold value data 502a to be referred to when a stage, in which the subject working on lifestyle habit improvement is, is judged.

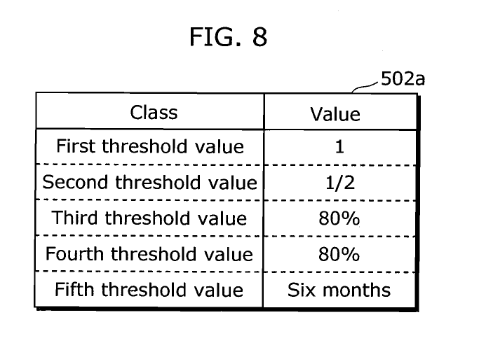

FIG. 8 is a drawing showing a first example of the threshold value data 502a stored in the threshold value storing unit 502. The threshold value data 502a exemplified in the drawing has a “threshold value” of which content is a threshold value to judge whether or not the subject belongs to a stage, and a “class” of which content is information showing an association of the “threshold value” with a stage judged with a value stored in the “threshold value”. The value stored in the “threshold value” includes a number, ratio, and duration. The threshold value data 502a in the embodiment, for example, represents the “class” of a threshold value for the first stage to be a “first threshold value”, and indicates the “value” to be “1”.

Here goes back to the description of FIG. 7.

The first stage judging unit 503, the second stage judging unit 504, the third stage judging unit 505, the fourth stage judging unit 506, and the fifth stage judging unit 507 are an example of a stage judging unit judging a stage at which the subject working on lifestyle habit improvement is, based on either the measurement value obtained by the measurement value obtaining unit 501, or the number of measurements representing the number of pieces of data of the measurement value obtained by the measurement value obtaining unit 501.

The first stage judging unit 503 judges whether the stage, at which the subject working on lifestyle habit improvement is, is the first stage, the second stage judging unit 504 judges whether the stage, at which the subject working on lifestyle habit improvement is, is the second stage, the third stage judging unit 505 judges whether the stage, at which the subject working on lifestyle habit improvement is, is the third stage, the fourth stage judging unit 506 judges whether the stage, at which the subject working on lifestyle habit improvement is, is the fourth stage, and the fifth stage judging unit 507 judges whether the stage, at which the subject working on lifestyle habit improvement is, is either the fifth stage, or the sixth stage. Each of the first stage judging unit 503, the second stage judging unit 504, the third stage judging unit 505, the fourth stage judging unit 506, and the fifth stage judging unit 507 shall be described in detail, hereinafter.

The first stage judging unit 503 obtains a threshold value corresponding to a “first threshold value” which the threshold value storing unit 502 stores (hereinafter referred to as a “first threshold value”). The first stage judging unit 503 is an example of a unit comparing the number of measurements and the first threshold value, and judging a stage, at which the subject working on lifestyle habit improvement is, to be the first stage based on a result of the comparison. Here, the number of measurements represents the number of pieces of data of the measurement value which the measurement value obtaining unit 501 obtains.

An example of the first stage is the “precontemplation stage”, which the subject is not interested in lifestyle habit improvement, for example.

Specifically, the first stage judging unit 503 is an example of a unit comparing the number of measurements and the first threshold value, and judging a stage to be the precontemplation stage in the case where the number of measurements is below the first threshold value. Here, the stage represents a stage, at which the subject working on lifestyle habit improvement is, as a stage in which the subject is not interested in lifestyle habit improvement.

The second stage judging unit 504 obtains a threshold value corresponding to a “second threshold value” which the threshold value storing unit 502 stores (hereinafter referred to as a “second threshold value”). The second stage judging unit 504 is an example of a unit comparing the target number of measurements with a figure to be obtained by multiplying the number of measurements by the second threshold value, and judging a stage, at which the subject working on lifestyle habit improvement is, to be the second stage based on a result of the comparison. Here, the number of measurements represents the number of pieces of data of the measurement value which the measurement value obtaining unit 501 obtains.

An example of the second stage is the “contemplation stage”, which the subject begins to be interested in lifestyle habit improvement, for example.

Specifically, the second stage judging unit 504 is an example of a unit: comparing the number of measurements with a figure to be obtained by multiplying the target number of measurements by the second threshold value; and judging a stage to be the contemplation stage in the case where the figure to be obtained, by multiplying the target number of measurements by the second threshold value, is equal to or less than the number of measurements. Here, the stage represents a stage, at which the subject working on lifestyle habit improvement is, as a stage in which the subject begins to be interested in lifestyle habit improvement.

The third stage judging unit 505 obtains a threshold value corresponding to a “third threshold value” which the threshold value storing unit 502 stores (hereinafter referred to as a “third threshold value”). The third stage judging unit 505 is an example of a unit: comparing the third threshold value with ratio of the number of pieces of data to the number of measurements; and judging a stage, at which the subject working on lifestyle habit improvement is, to be the third stage based on a result of the comparison. Here, the number of pieces of data has a discrepancy degree, to be equal to or less than a certain value, between the measurement value and the probable value which the measurement value obtaining unit 501 obtains.

An example of the third stage is the “first preparation stage”, which the subject begins working on lifestyle habit improvement but has not acquired correct knowledge, for example.

Specifically, the third stage judging unit 505 is an example of a unit: comparing the third threshold value with ratio of the number of pieces of data to the number of measurements; and judging a stage, at which the subject working on lifestyle habit improvement is, to be the first preparation stage in which the subject starts working on lifestyle habit improvement but does not acquired correct knowledge is, in the case where the ratio of the number of pieces of data to the number of measurements is smaller than the third threshold value. Here, the number of pieces of data has the discrepancy degree, between the measurement value and the probable value, which is equal to or less than a certain value.

The fourth stage judging unit 506 obtains a threshold value corresponding to a “fourth threshold value” which the threshold value storing unit 502 stores (hereinafter referred to as a “fourth threshold”). The fourth stage judging unit 506 is an example of a unit: comparing the fourth threshold value with ratio of the number of pieces of data to the number of measurements; and judging a stage, at which the subject working on lifestyle habit improvement is, to be the fourth stage based on a result of the comparison. Here, the number of pieces of data indicates that the measurement value which the measurement value obtaining unit 501 obtains is equal to or less than the target measurement value.

An example of the fourth stage is the “second preparation stage”, which the subject begins working on lifestyle habit improvement but is short of utilizing acquired correct knowledge, for example.

Specifically, the fourth stage judging unit 506 is an example of a unit: comparing the fourth threshold value with ratio of the number of pieces of data to the number of measurements; and judging a stage, at which the subject working on lifestyle habit improvement is, to be the second preparation stage in which the user is short of utilizing the acquired correct knowledge, in the case where the ratio of the number of pieces of data to the number of measurements is smaller than the fourth threshold value. Here, the number of pieces of data indicates that the measurement value is equal to or less than the target number of measurements.

The fifth stage judging unit 507 obtains the fourth threshold value and a threshold value corresponding to a “fifth threshold” which the threshold value storing unit 502 stores (hereinafter referred to as a “fifth threshold”). The fifth stage judging unit 507 is an example of a unit: comparing the fifth threshold value with duration; and judging a stage, at which the subject working on lifestyle habit improvement is, to be the fifth or sixth stage based on a result of the comparison. Here, the duration represents ratio of the number of pieces of data to the number of measurements to be greater than the fourth threshold value, the number of pieces of data indicating the measurement value which the measurement value obtaining unit 501 obtains to be equal to or less than the target measurement value.

An example of the fifth stage is the “action stage”, which the subject begins working on lifestyle habit improvement, utilizing correct knowledge, for example.

Specifically, the fifth stage judging unit 507 is an example of a unit: comparing the duration and the fifth threshold value; and judging a stage, at which the subject working on lifestyle habit improvement is, to be the action stage in which the subject starts working on lifestyle habit improvement, utilizing the correct knowledge, in the case where the duration is smaller than the fifth threshold value. Here, the duration represents the ratio of the number of pieces of data to the number of measurement times to be greater than the fourth threshold, the number of pieces of data indicating the measurement value to be equal to or less than the target measurement value.

An example of the sixth stage is the “maintenance stage”, which an appropriate lifestyle habit has been established for the health of the subject, for example.

Specifically, the fifth stage judging unit 507 is an example of a unit: comparing the duration with the fifth threshold value; and judging a stage, at which the subject working on lifestyle habit improvement is, to be the maintenance stage in which an appropriate lifestyle habit has been established for the health of the subject, in the case where the duration is equal to or more than the fifth threshold. Here, the duration represents the ratio of the number of pieces of data to the number of measurement times to be greater than the fourth threshold, the number of pieces of data indicating the measurement value to be equal to or less than the target measurement value.

It is noted that all of the measurement value, the target measurement value, and the target number of measurements are assumed to be stored in a single storing medium 2 in the embodiment; meanwhile, the measurement value, the target measurement value, and the target number of measurements may be stored in a different storing medium. For example, the target value and the target measurement value may be stored in a storing unit (not shown) included in the lifestyle habit improvement supporting apparatus 4.

Referring to the stage in the stage instruction content table stored in the instruction content storing unit 409, the instruction deciding unit 509 decides an instruction content suitable for a judgment result obtained from the first stage judging unit 503, the second stage judging unit 504, the fourth stage judging unit 1805, and the fifth stage judging unit 507.

In other words, the instruction deciding unit 509 is an example of an instruction deciding unit deciding an instruction method and advice in based on a stage at which the subject working on lifestyle habit improvement is, referring to: a table storing a stage at which the subject working on lifestyle habit improvement is; and the instruction method and advice based or the stage at which the subject working on the lifestyle habit improvement is, in accordance with the judgment result by the stage judging unit.

The display controlling unit 510 controls the displaying unit 403. The displaying unit 510 in the embodiment causes the displaying unit 403 to display information on the subject, such as the stage at which the subject working on lifestyle habit improvement is and the instruction content obtained from the instruction deciding unit 509, and the subject's name.

To be more specific, the display controlling unit 510 is: an example of a first displaying controlling unit to cause at least one of the judgment result by the stage judging unit and an instruction method or advice decided by the instruction deciding unit to be displayed; and an example of a second display controlling unit to cause the measurement value, the probable value, and the target value for a certain number of measurements to be displayed.

Furthermore, a flow of urine component measurement, using the urine component measuring instrument 1, shall be described referring to FIG. 9.

FIG. 9 is a flowchart of processing executed by the urine component measuring instrument 1 in the first embodiment.

First, the CPU 106 judges whether or not the power button 101 is pressed (S101). When judging the power button 101 not to be pressed (S101: No), the CPU 106 stands by until the power button 101 is pressed.

When judging the power button 101 to be pressed (S101: Yes), the CPU 106 causes the displaying unit 103 to display an input request of a probable value (S102).

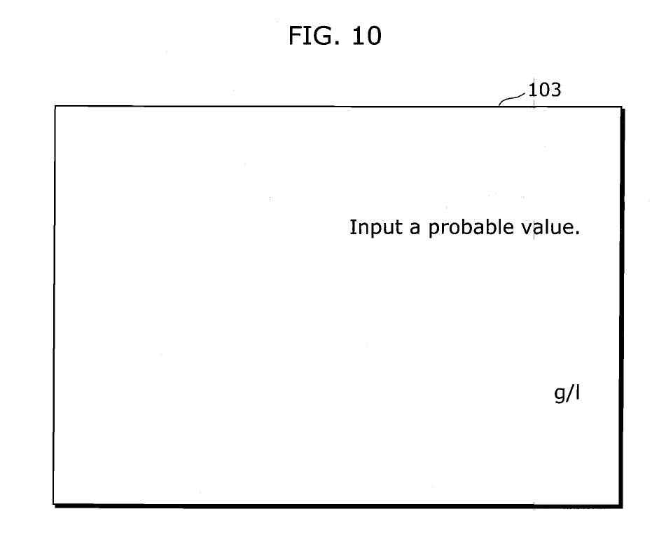

Here, FIG. 10 is a drawing showing an example of an input request of a probable value displayed on the displaying unit 103 in the urine component measuring instrument 1. The displaying unit 103 exemplified in the drawing displays an input request “Input a probable value”.

The subject inputs a probable value of salinity concentration in urine assumed out of his or her diet into the urine component measuring instrument 1 on the input unit 102.

Here goes back to the description of the FIG. 9.

Next, the CPU 106 judges whether or no the probable value is inputted (S103). When judging the probable value not to be inputted (S103: No), the CPU 106 stands by until the probable value is inputted.

When judging the probable value to be inputted (S103: Yes), the CPU 106 causes the displaying unit 103 to display an attachment request of the measuring chip 3 (S104).

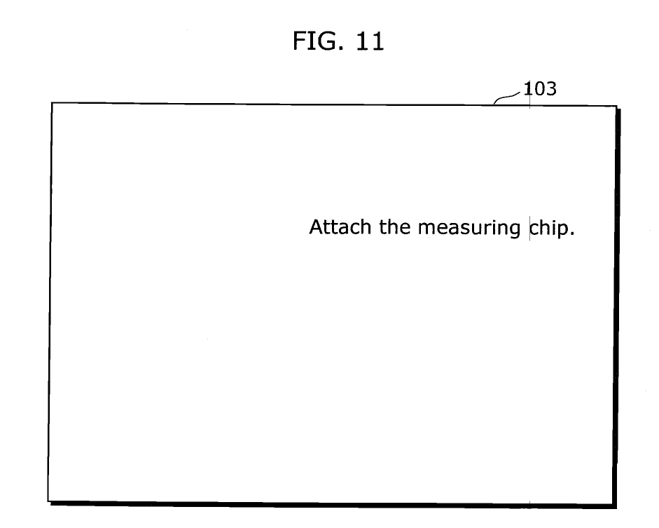

Here, FIG. 11 is a drawing showing an example of the attachment request of the measuring chip 3 displayed on the displaying unit 103 in the urine component measuring instrument 1. The drawing exemplifies the attachment request of the measuring chip 3 “Attach the measuring chip” to be displayed.

Here goes back to the description of the FIG. 9.

Then, based on the fact whether or not the attachment detecting unit 109 has received a notification that the attachment detecting unit 109 has detected the attachment of the measuring chip 3, the CPU 106 judges whether or not the measuring chip 3 is attached to the measuring chip connecting port 105 (S105).

When not receiving the notification from the attachment detecting unit 109 that the measuring chip 3 has been attached, the CPU 106 judges the measuring chip 3 not to be attached (S105: No), and stands by until receiving the notification from the attachment detecting unit 109 that the measuring chip 3 has been attached.

When receiving the notification from the attachment detecting unit 109 that the measuring chip 3 has been attached, the CPU 106 judges the measuring chip 3 to be attached (S105: Yes), and causes the displaying unit 103 to display a message notifying that the measurement preparation is ready (S106).

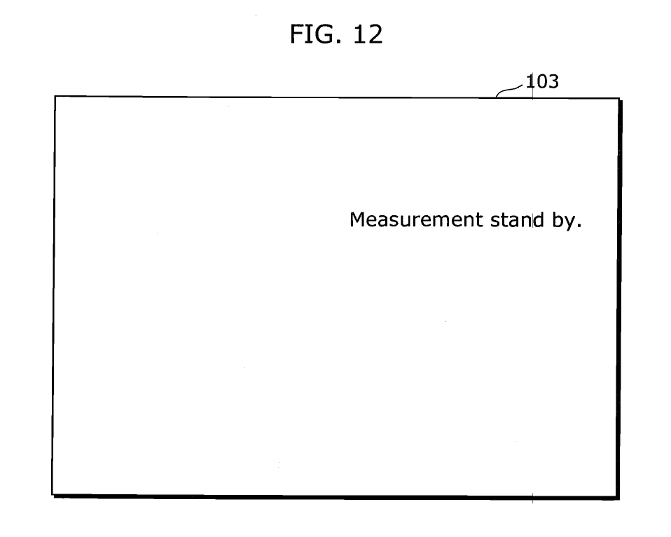

Here, FIG. 12 is a drawing exemplifying a message notifying that the measurement preparation, displayed on the displaying unit 103 in the urine component measuring instrument 1 of the first embodiment, is ready. This drawing is an example of displaying a notifying message “Measurement stand by” showing that the measurement preparation is ready.

Here goes back to the description of the FIG. 9.

The subject put a portion of the electrode 301 of the measuring chip 3 attached to the urine component measuring instrument 1 in urine which the subject has sampled in a container, such as a paper cup.

Further, the CPU 106 judges whether or not some sort of input has been performed into the input unit 102 (S107). When judging that nothing has been inputted into the input unit 102 (S107: No), the CPU 106 stands by in the state of the measurement preparation.

When judging that some sort of input has been performed, (S107: Yes), the CPU 106 measures the salinity concentration in the urine (S108). Specifically, the CPU 106: refers to a measurement table, showing relationship between a value of resistance and salinity concentration, stored in the storing medium 2; and converts the value of resistance to salinity concentration to measure the salinity concentration in the urine. It is noted that the measurement table may be stored in a storing unit included in the urine component measuring instrument 1 (not shown).

Moreover, the CPU 106 causes the displaying unit 103 to display a measurement value, which is the salinity concentration in the measured urine, for a certain period of time (S109).

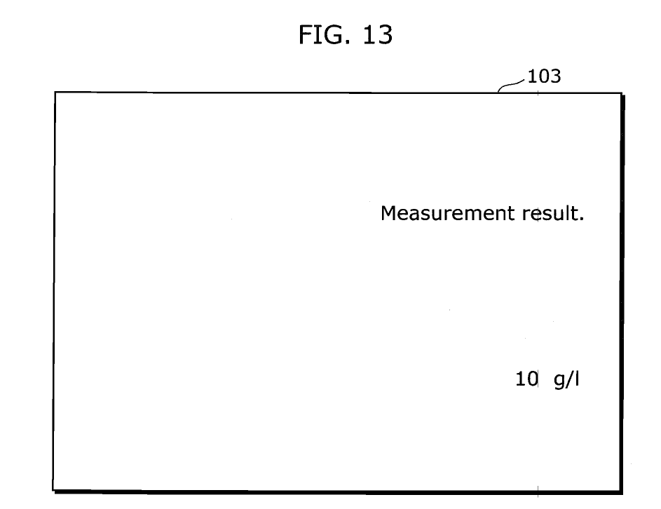

Here, FIG. 13 is a drawing exemplifying a measurement value displayed on the displaying unit 103 in the urine component measuring instrument 1 of the first embodiment. This drawing shows an example of the salinity concentration in the measured urine to be “10 g/l”.

Then, the CPU 106 links, to store into the storage medium 2, a measurement date and time obtained from the timer 108, the result measured in S108, and the probable value inputted based on the input request displayed in S102 (S110).

Further, the CPU 106 causes the displaying unit 103 to display: the probable value, probable values which are identical to the target value and the measurement value, the target value and the measurement value which are stored in the storing medium 2 in processing of S110; and a probable value, a target value, and a measurement value which have been previously measured and stored in the storing medium 2.

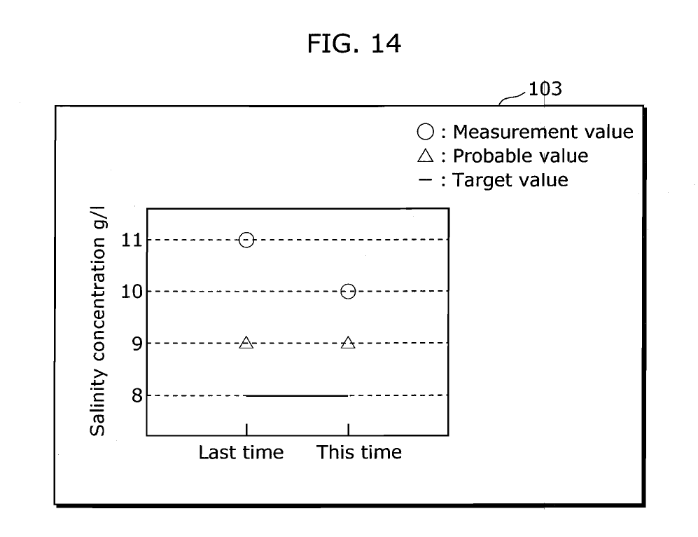

Here, FIG. 14 is a drawing exemplifying the probable value, the target value, and the measurement value displayed on the displaying unit 103 in the urine component measuring instrument 1 of the first embodiment. The urine component measuring instrument 1 in the embodiment displays the probable value, the target value and the measurement value stored in the storing medium 2 in a graph.

This display allows the subject to compare the last time and this time at a glance whether or not the probable value and the measurement value develop a discrepancy, or the target value and the measurement value develop a discrepancy, so that a stage on which the subject works on lifestyle habit can be objectively and easily recognized.

Here goes back to the description of the FIG. 9.

Finally, the CPU 106 judges whether or not the power button is pressed (S111). In the case where the power button 106 is not pressed (S112: No), the CPU 106 continue the display processing in S110.

In the case where, the power button 106 is pressed (S112: Yes), the CPU 106 shuts down the power 107 to finish the processing.

As mentioned above, under an instruction of an expert, such as a doctor, the subject measures salinity in urine, working on lifestyle improvement in a daily life. In the case of a subject receiving a regular instruction from a doctor, for example, the subject brings the storing medium 2 which a measurement value is recorded when receiving medical care at a medical institution once a month, for example. Using the lifestyle habit improvement supporting apparatus 4 in a clinic, the doctor reads and analyzes the subject's measurement value to conduct a life improvement instruction based on a stage of the subject.

In the case of salinity measurement in urine, the subject preferably takes the measurement once a day upon getting up.

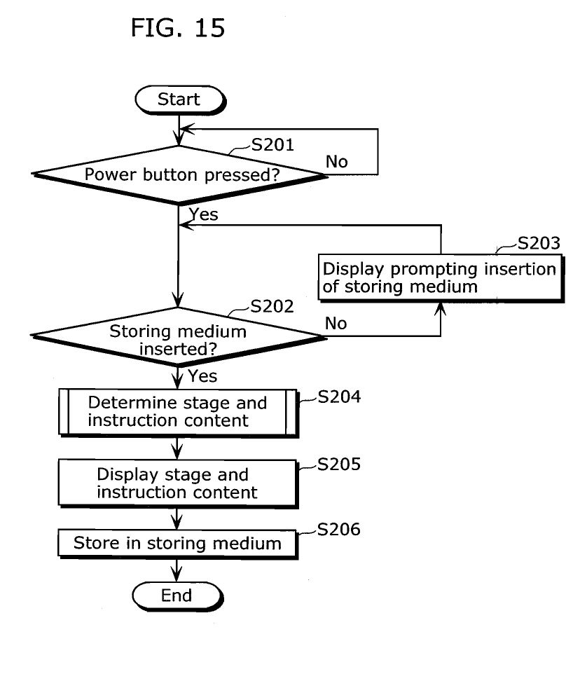

Further, a determination of a stage and an instruction content in the lifestyle habit improvement supporting apparatus 4 of the first embodiment shall be described referring to FIG. 15 through FIG. 18.

FIG. 15 is a flowchart showing a flow of processing executed on the lifestyle habit improvement supporting apparatus 4 in the first embodiment. The flowchart exemplifies a case where the subject measures everyday his or her salinity concentration in urine using the urine component measuring instrument 1, and performs once a month a determination of the result by the lifestyle habit improvement supporting apparatus 4 under a control of a lifestyle habit improvement instructor.

First, the CPU 404 judges whether or not the power button is pressed (S201). When judging the power button not to be pressed (S201: No), the CPU 404 stands by until the power button is pressed.

When judging the power button to be pressed (S201: Yes), then the CPU 404 judges whether or not the storing medium 2 is inserted into the storing medium insertion slot 402 (S202).

When judging the storing medium 2 not to be inserted, (S202: No), the CPU 404 displays a message, on the displaying unit 403, for prompting insertion of the storing medium 2.

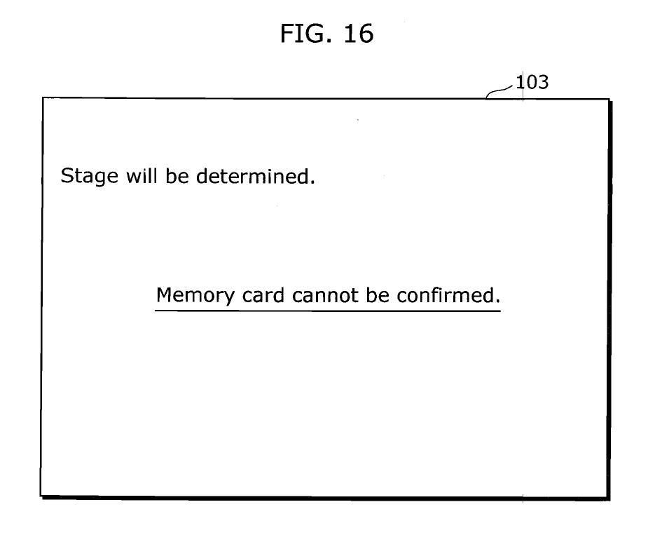

FIG. 16 is a display example of a message, for prompting insertion of the storing medium 2, displayed on the displaying unit 403 in the lifestyle habit improvement supporting apparatus 4 in the first embodiment. This is an example that by causing the display unit 403 to display a message “Memory card cannot be confirmed”, the CPU 404 prompts the user to insert a storing medium.

When judging the storing medium 2 to be inserted, (S202: Yes), the CPU 404 reads a stage determining program out of the determination program storing unit 408 and execute the program to determine a stage at which the subject is and the instruction content based on the information which the storing medium 2 stores (S204). Details of determination processing of the stage and the instruction content shall be described hereinafter.

Then, the CPU 404 causes the displaying unit 403 to display: the stage, at which the subject is, determined in S204, and the instruction content suitable for the stage obtained by referring to the stage instruction content table stored in the instruction content storing unit 409 (S205).

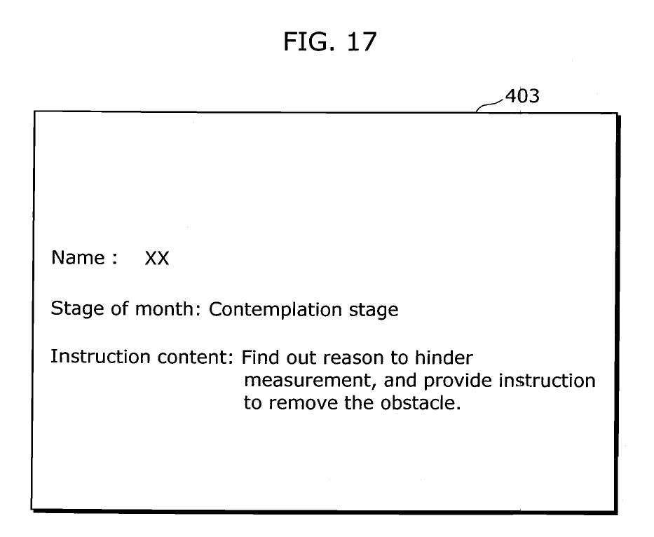

Here, FIG. 17 exemplifies a display of the stage at which the subject is and the instruction content displayed on the displaying unit 403 in the lifestyle habit improvement supporting apparatus 4 of the first embodiment.

On the displaying unit 403, is displayed the following: the subject's “name”, a determined “stage of this month”, and an “instruction content” suitable for the stage. The stage, at which the subject is, determined in the drawing is the “contemplation stage”.

In addition, the drawing is an example: in the case where the instruction content storing unit 409 stores the stage instruction content table shown in FIG. 5; and displaying, as an instruction content, the “instruction content” suitable for the “contemplation stage”, that is, “Find out a reason to hinder measurement, and provide an instruction to remove the obstacle”.

Next, the CPU 404 links, to store into the storing medium 2, the stage, at which the subject is, determined in S204, an end-of-determination time obtained from the timer 407, and the “action stage and maintenance stage duration (month)”, and ends the processing (S206).

Information to be stored in the “action stage and maintenance stage duration (month)” indicates duration which the “action stage and maintenance stage duration (month)” of the previous determination added to the elapsed time from the previous determination makes, in the case where the determination this time is either the “action stage”, or the “maintenance stage”. The information stored in the “action stage and maintenance stage duration (month)” can be calculated by referring to the “action stage and maintenance stage duration (month)” stored at the previous determination. In the case where a determination is performed once a month, the information is duration in which one month is added to the “action stage and maintenance stage duration (month)” of the previous determination.

In addition, this time of the determination is other than the “action stage” and the “maintenance stage”, the information stored in the “action stage and maintenance stage duration (month)” is “0”.

Next, details of determination processing, of a stage and an instruction content, which the CPU 404 executes shall be described, referring to FIG. 7 and FIG. 18.

FIG. 18 is a flowchart showing a first example of details of processing which the CPU 404 executes in the stage and instruction content determining processing S204 shown in FIG. 15. More specifically, the drawing is: an example of processing which the first program execution CPU 404 executes; and a flowchart showing the case where the subject measures his or her salinity concentration in urine everyday, using the urine component measuring instrument 1, and performs once month the determination of the result by the lifestyle habit improvement supporting apparatus 4 under the control of the lifestyle habit improvement instructor.

First, the measurement value obtaining unit 501 extracts information on the subject's lifestyle habit improvement over the past one month from the storing medium 2. (S301).

Next, the first stage judging unit 503 obtains the number of measurements from the number of pieces of the measurement data extracted from S301, and then compares the number of measurements and the first threshold value. In the example shown in the drawing, whether or not the measurement data extracted in S301 is judged (S302), and the first threshold is set to “1”. In the case where a judgment is made that no measurement data over the past one month exists, (S302: No), the first stage judging unit 503 judges the stage at which the subject is as the “precontemplation stage” (S303). Obtaining the fact that the judgment of the first stage judging unit 503 is the “precontemplation stage” and referring to the stage instruction content table stored in the instruction content storing unit 409, the instruction deciding unit 509 decides an instruction content suitable for the “precontemplation stage”.

When judging the number of pieces of the measurement data over the past one month is one or more, (S302: Yes), then, the second stage judging unit 504 sets the number of measurement performed by the subject over the past one month to A (S305). Here, the extracted number of measurement can be obtained from the number of pieces of measurement data over the past one month. Referring to the target number of measurements included in the information on lifestyle habit improvement stored in the storing medium 2, the second stage judging unit 504 judges whether or not A is greater than a value which the target number of measurements multiplied by the second threshold makes. In the example shown in the drawing, whether or not A is greater than a value that the target number of measurements multiplied by ½ is judged (S306), and the second threshold value is set to “½”.

When A is judged not to be greater than ½ of the target number of times (S306: No), the subject, showing some sort of interest in measurement, has achieved only half of the target number which the lifestyle habit improvement instructor has instructed to achieve. Thus, the second stage judging unit 504 determines the stage at which the subject is to be the “contemplation stage”. Referring to the stage instruction content table stored in the instruction content storing unit 409, the instruction deciding unit 509 decides an instruction content suitable for the “contemplation stage” (S308).

When A is judged to be greater than ½ of the target number of measurements, (S306: Yes), then, the third stage judging unit 505 calculates ratio of a measurement to the number of measurements over the past one month, out of the extracted measurement data over the past one month, to be B. Here, each measurement has a discrepancy degree, between the probable value and the measurement value, to be equal to or less than certain ratio. In the example shown in the drawing, ratio of a measurement to the number of measurements over the past one month is calculated, out of the extracted measurement data over the past one month, to be B (S309). Here, each measurement has a discrepancy degree, between the probable value and the measurement value, to be equal to or less than 20 percent.

Next, the third stage judging unit 505 judges whether or not B is equal to or more than the third threshold. In the example shown in the drawing, whether or not B is equal to or more than 80 percent is judged (S310), and the third threshold value is set to be “80%”.

In the case where B is judged to be below 80 percent (S310: No), the following is assumed: the subject is acknowledged to be interested in the improvement since working on the measurement equal to or more than the target number of measurements; meanwhile the subject lacks knowledge on amount of salt in an actual diet since a large discrepancy between the probable value and the measurement value is observed. Thus, the third stage judging unit 505 judges the stage at which the subject is to be the “first preparation stage” (S311). Referring to the stage instruction content table stored in the instruction content storing unit 409, the third stage judging unit 505 decides an instruction content suitable for the “first preparation stage” (S312).

When judging B to be equal to or more than 80 percent, (S310: Yes), then, the fourth stage judging unit 506 calculates ratio of a measurement to all the number of measurements over the past one month, out of the extracted measurement data over the past one month, to be C (S313). Here, each measurement has a measurement value being equal to or less than the target value.

Next, the fourth stage judging unit 506 judges whether or not C is equal to or more than the fourth threshold value. In the example shown in the drawing, whether or not C is equal to or more than 80 percent is judged (S314), and the fourth threshold value is set to be “80%”.

In the case where C is judged to be below 80 percent (S314: No), the following is assumed: the subject is eager to work on for lifestyle habit improvement and has knowledge and motivation to comprehend the amount of salt in his or her diet; meanwhile, the subject is in a state short of knowledge and know-how on reducing amount of salt in diet. Thus, the fourth stage judging unit 506 judges the stage, at which the subject is, to be the “second preparation stage” (S315). Referring to the stage instruction content table stored in the instruction content storing unit 409, the fourth stage judging unit 506 decides an instruction content suitable for the “second preparation stage” (S316).

When judging C to be 80 percent or above (S314: Yes), the fifth stage judging unit 507 refers to the “action stage and maintenance stage duration (month)”, included in the information on lifestyle habit improvement stored in the storing medium 2, to set a period stored in the “action stage and maintenance stage duration (month) to D (S317).

Following which, the fifth stage judging unit 507 judges whether or not D is equal to or more than the fifth threshold. In the example shown in the drawing, the fifth stage judging unit 507 judges whether or not D is equal to or longer than six months (S318), and sets the fifth threshold value to “six month”.

When judging D to be shorter than six month, (S318: No), the fifth stage judging unit 507 judges the stage at which the subject is to be the “action stage” (S319). When judging D to be equal to or longer than six months, (S318: Yes), the fifth stage judging unit 508 judges the stage at which the subject is to be the “action stage” (S320). Then, referring to the stage instruction content table stored in the instruction content storing unit 409, the instruction deciding unit 509 decides an instruction content suitable for each of the “action stage”, and the “maintenance stage” (S321).

As mentioned above, the first program execution CPU 404 decides an instruction content appropriate to the subject. The lifestyle habit improvement instructor provides an instruction to the subject based on the instruction content displayed in S205 of FIG. 15.

In the case where a stage is determined to be the “precontemplation stage”, the instructor provides an instruction to raise an interest in lifestyle habit improvement and knowledge of illness.

In the case where a stage is judged to be the “contemplation stage”, the instructor thinks are son why the subject cannot carry out the measurement even though interested in the measurement, and provides an instruction to remove the obstacle. Specifically, the instruction is to present benefit which the subject enjoys when carrying out measurement and drawback which the subject suffers when neglecting measurement.

In the case where a stage is judged to be the “first preparation stage”, the instructor intensively provides an instruction on a calculation method of salt out of a taken meal. To be specific, the instructor presents a method for: estimating the amount of salt with a food administration table; and remembering details of a meal.

In the case where a stage is judged to be the “second preparation stage”, the instructor proposes a specific method for improvement in order to cut the amount of salt in a meal. In such case, the subject's lifestyle habit can be efficiently improved not only by just reducing the amount of salt in the meal, but also by presenting a tip to avoid making the meal tasteless, as well.

Finally, in the case where a stage is judged to be either the “action stage” or the “maintenance stage”, the instructor may focuses an instruction on how to keep the subject's motivation for the lifestyle habit improvement. The instruction is, for example, to develop the subject's surrounding environment by seeking the subject's family help, so that the subject will not reverse the lifestyle habit improvement to which the subject get accustomed once.

Furthermore, referring to FIGS. 15 and 18, a specific example of processing by the first program execution CPU 404 shall be described with regard to the details of the determination processing of the stage and the instruction content (S204), the display processing of the instruction content (S205), and the storing processing of the stage and the instruction content into a storing medium (S206). Here, the specific example is provided in the case where the stage instruction content table stored in the instruction content storing unit 409 represents the content shown in FIG. 5, and the information on lifestyle habit improvement stored in the storing medium 2 represents the content shown in FIG. 6. The date and time when the stage determination is executed is “10:00, Oct. 1, 2004”, and a cycle of a lifestyle habit improvement instruction is one month.

First, in the determination processing of the stage and the instruction content (S204), the first program execution CPU 404 extracts the data over the past one month from the storing medium 2 (S301). In the specific example, the “measurement date and time” is measurement data from Sep. 1, 2004 to Sep. 30, 2004. Measurement information shown “2” through “9” in the “data number” in FIG. 6 is extracted.

Next, the first stage judging unit 503 judges whether or not the measurement data exists (S302). Since the measurement data exists in the specific example as mentioned above, the first stage judging unit 503 judges the measurement data to exist (S302: Yes).

Then, the second stage judging unit 504 sets the number of pieces of the measurement data to A (S305). In the specific example, the number of pieces of the measurement data is 8, and thus A is set to 8.

Further, the second stage judging unit 504 refers to the target number of measurements to judge whether or not A is larger than a value that the target number of measurement is divided by two (S306). In the specific example, the target number of measurements is “30” as shown in FIG. 6, and thus, the value divided by two is 15. Since A is set to 8, the second stage judging unit 504 judges that A is not greater than the value that the target number of measurements is divided by two (S306: No).

Then, the second stage judging unit 504 judges the “stage” in the specific example to be the “contemplation stage” (S307).

Further, the instruction deciding unit 509 refers to the instruction content storing unit 409 shown in FIG. 5 to decide the instruction content; that is, “Find out a reason to hinder measurement, and provide an instruction to remove the obstacle”.

Next, the display controlling unit 510 causes the displaying unit 403 to display the determined stage and instruction (S205). The above FIG. 17 exemplifies the result of the above processing to be displayed on the displaying unit 403.

Then, the first program execution CPU 404 stores the determined stage into the storing medium 2 (S206) and finishes the processing. The information shown with “10” in the “data number” exemplifies the judgment result in the specific example to be stored. Here, the “stage” to be determined is the “contemplation stage”, so the “action stage and maintenance stage duration (month)” is set to “0”.

As mentioned above, the lifestyle habit improvement supporting apparatus 4 in the first embodiment can evaluate a state of the subject (stage) based on a theory of behavioral science in accordance with objective measurement data, so that a stage at which the subject is can be accurately determined. Thus, a doctor can accurately comprehend the stage to provide a more accurate instruction to the subject.

It is noted that in the embodiment, information on the subject's lifestyle improvement has been given and received via a storing medium; namely, a memory card between the urine component measuring instrument 1 and the lifestyle habit improvement supporting apparatus 4. The storing medium is not limited to a memory card. For example, a flexible disk, an MO (Magnet-Optical disk), a USB (Universal Serial Bus) memory may also be utilized.

Further, the storing medium may be a memory and a hard disk provided in the apparatus, other than a dismountable memory card from the apparatus. In this case, giving and receiving information between the urine component measuring instrument 1 and the lifestyle habit improvement supporting apparatus 4 is replaced by transmitting and receiving data, with wired or wireless connection between the urine component measuring instrument 1 and the lifestyle habit improvement supporting apparatus 4.

Further, the embodiment uses the terminal for storing medium 410 provided in the lifestyle habit improvement supporting apparatus 4 as a scheme to input the data stored in the storing medium 2 into the lifestyle habit improvement supporting apparatus 4; however, the scheme is not limited to this. For example, instead of the terminal for storing medium 410, a cellular phone and a PDA (Personal Digital Assistance) including a reader of the storing medium 2 may also be used. By attaching the storing medium to the reader, and utilizing wireless communication on the cellular phone and the PDA terminal with the storing medium attached, a measurement value is transmitted to a receiving unit (not shown) provided in the lifestyle habit improvement supporting apparatus 4. Here, since the measurement value is personal information of the subject, the data is desirably encrypted to be transmitted.

Moreover, the data between the urine component measuring instrument 1 and the lifestyle habit improvement supporting apparatus 4 may be given and received via a public circuit network.

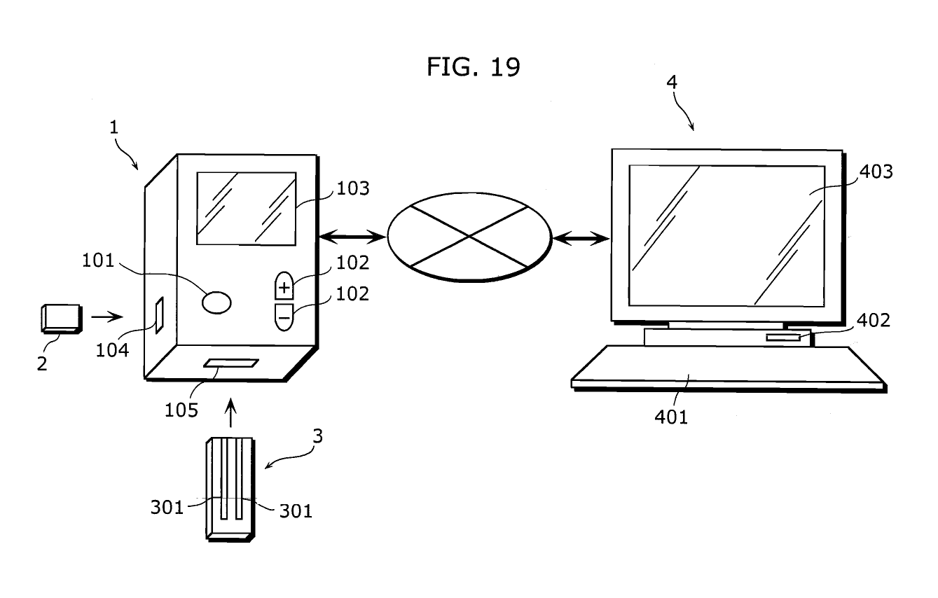

FIG. 19 is a schematic view showing a first modification example of the configuration of the lifestyle habit correction supporting system in the first embodiment. The urine component measuring instrument 1 and a terminal for clinic 4 each includes a data transmitting and receiving unit and mutually transmits and receives data via the public circuit network.

This allows a risk, such as losing a storing medium, to be avoided since the storing medium 2 does not need to be carried around for data transmission and reception. Here, either a wired or a wireless connection scheme may be utilized between the urine component measuring instrument 1 and the terminal for clinic 4.

Further, in the embodiment, the number of measurements, the measurement value, the target value, and the probable value are used in the case of determining a stage; however, the probable value may be omitted in the configuration. In this case, a stage at which the subject is can be determined by determining the “first preparation stage” and the “second preparation stage” shown in the embodiment into a single stage; namely the “preparation stage”, for example.

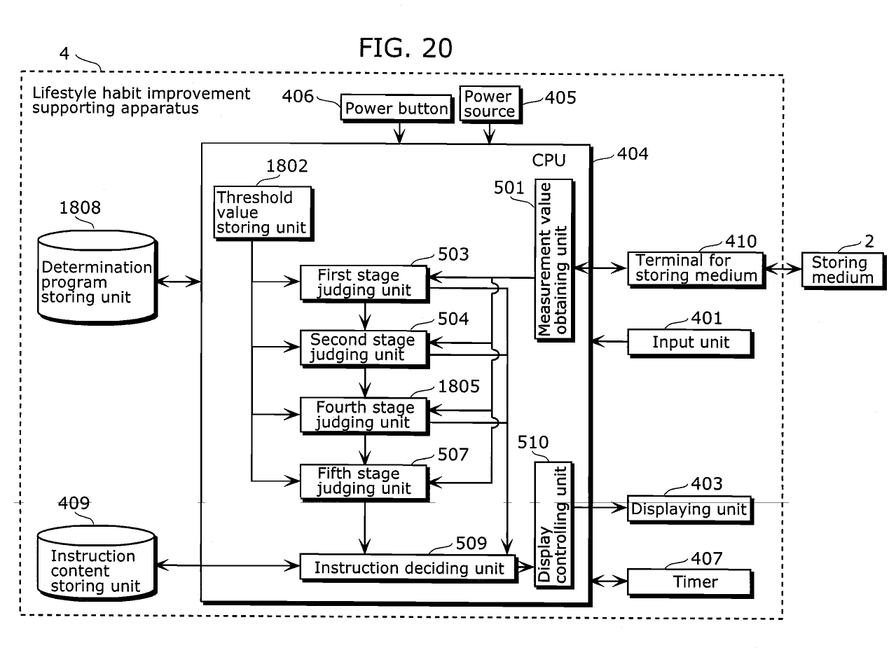

FIG. 20 is a block diagram showing a configuration of a function performed by the CPU 404 executing a second program stored in a determination program storing unit 1803 in a second modification of the first embodiment. The CPU 404 executing the second program which the determination program storing unit 1808 stores (referred to as a “second program execution CPU 404” hereinafter) determines a stage in which the subject works for lifestyle improvement out of the measurement value of the subject's the salinity concentration in urine stored in the storing medium 2, and then provides an instruction outline based on the determination.

Here, the six stages are determined by the first program execution CPU 404; namely, the “precontemplation stage”, the “contemplation stage”; the “first preparation stage”, the “second preparation stage”, the “action stage”, and the “maintenance stage”. Instead, five stages are determined by the second program execution CPU 404; namely, “precontemplation stage”, the “contemplation stage”, the “preparation stage”, the “action stage”, and the “maintenance stage”.

The second program execution CPU 404 includes the measurement value obtaining unit 501, the threshold value storing unit 1802, the first stage judging unit 503, the second stage judging unit 504, the fourth stage judging unit 1805, the fifth stage judging unit 507, the instruction deciding unit 509, and the display controlling unit 510. The units, described by referring to FIG. 7, having the same function as those in the first program execution CPU 404 have the same numerical marks, and thus, the description shall be omitted here.

Further, the lifestyle habit improvement supporting apparatus 4: has the following out of the CPU 404; namely, the input unit 401, the displaying unit 403, the power source 405, the power button 406, the timer 407, the determination program storing unit 1808 which stores the second program, the instruction content storing unit 409, and the terminal for storing medium 410 which is connection to the storing medium 2; and gives and receives information to and from these. The units the CPU 404 externally has have been described with reference to FIG. 4, and thus the description shall be omitted here.

The threshold value storing unit 1802 stores threshold value data 1802a to be referred to when a stage, in which the subject works on for lifestyle habit improvement, is determined.

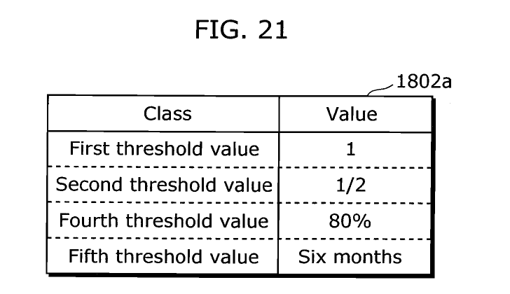

FIG. 21 is a drawing showing a second example of the threshold value data 1802a stored in the threshold value storing unit 1802. The structure of the threshold value data 1802a is the same as that of the threshold value data 502a stored in the threshold value storing unit 502 in the first program execution CPU 404 described with reference to FIG. 8, and thus the description here shall be omitted.

A difference between the threshold value data 502a in the first program execution CPU 404 and the threshold value data 1802a in the second program execution CPU 404 is that a “third threshold value” is included in the threshold value data 502a in the first program execution CPU 404; meanwhile, the “third threshold value” is not included in the threshold value data 1802a in the second program execution CPU 404.

The fourth stage judging unit 1805 obtains the fourth threshold value which the threshold value storing unit 1802 stores. The fourth stage judging unit 1805 is an example of a unit: comparing ratio of the number of pieces of data to the number of measurements with the fourth threshold value; and judging a stage, at which the subject working on lifestyle habit improvement is, to be the fourth stage based on a result of the comparison. Here, the number of pieces of data indicates that a measurement value which the measurement value obtaining unit 501 obtains is equal to or less than the target number of measurements.

In other words, the fourth stage judging unit 1805 is an example of a unit: comparing the fourth threshold value with ratio of the number of pieces of data to the number of measurements; and judging a stage, at which the subject working on lifestyle habit improvement is, to be the preparation stage in which the user has started working on lifestyle habit improvement, in the case where the ratio of the number of pieces of data, of which the measurement value is equal to or less than a target measurement value, to the number of measurements is smaller than the fourth threshold value. Here, the number of pieces of data indicates that the measurement value is equal to or less than the target measurement value.

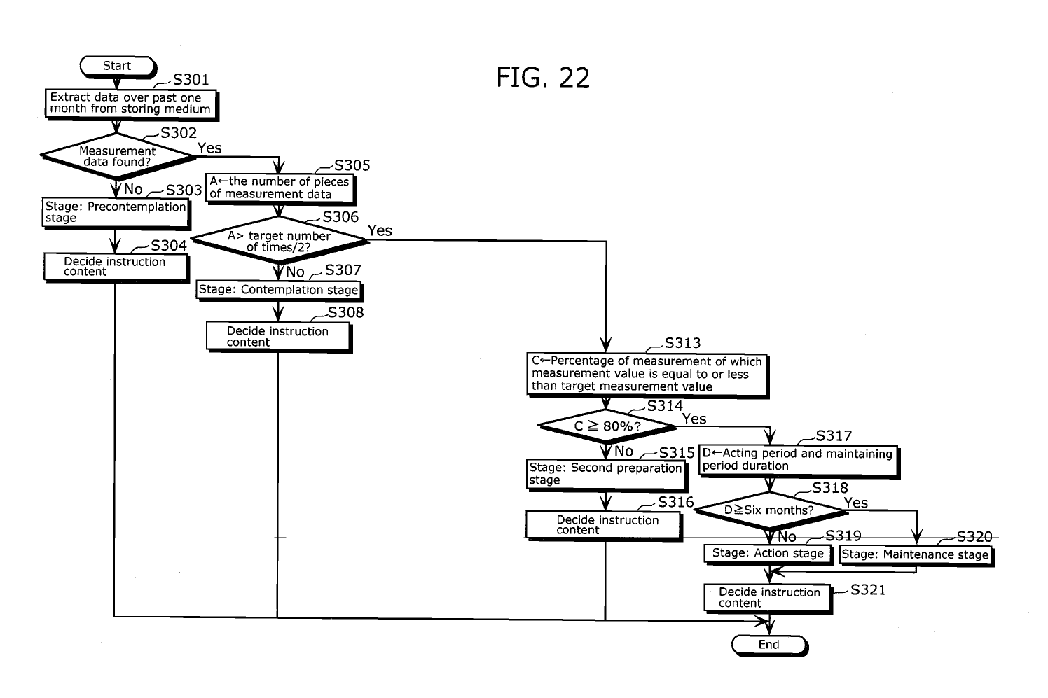

The FIG. 22 is a flowchart showing a second example of details which the CPU 404 executes in the stage and instruction content determination processing S204 shown in FIG. 15. The flowchart shown in the drawing illustrates processing in the second program execution CPU 404, which deletes processing from S309 to S312 from the flowchart shown in FIG. 18 and changes a result of the stage determination executed in S315 to the “preparation stage” (S3151). In addition, processing in S3151 is executed by a fourth stage judging unit 1805.

The numerical marks in the drawing are the same as those of the processing in the flowchart shown in FIG. 18. Thus, the description shall be omitted.

This allows processing of the subject inputting the probable value (S102 and S103 in FIG. 9) to be omitted, so that a stage determination giving a lessened burden to the subject is possible.

Moreover, a standard of judgment processing executed by the CPU 404 in S306, S310, and S318 shown in FIGS. 18 and 22 is not limited to the standard shown in the drawing. Setting of an appropriate judgment standard enables a stage determination of the subject to be accurately executed out of an objective value measured by the urine component measuring instrument 1.

### Second Embodiment

Next, a lifestyle habit improvement supporting apparatus in a second embodiment shall be described.

In the lifestyle habit improvement supporting apparatus of the embodiment, the urine component measuring instrument 1 of the first embodiment additionally includes a determination program storing unit and an instruction content storing unit. This allows a subject not only to measure his or her salinity concentration in urine but also to determine his or her stage, himself or herself.

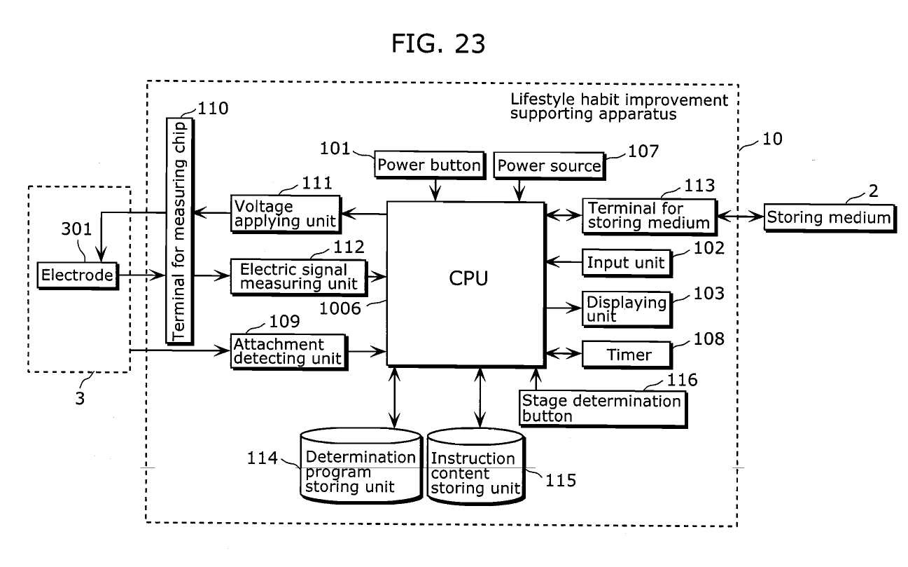

FIG. 23 is a block diagram showing a functional structure of a lifestyle habit improvement supporting apparatus 10 in the second embodiment. The lifestyle habit improvement supporting apparatus 10 in the second embodiment has the function of the urine component measuring instrument 1 in the first embodiment with the function of stage determination included.

The lifestyle habit improvement supporting apparatus 10 in the second embodiment includes the power button 101, the input unit 102, the displaying unit 103, the power source 107, the timer 108, the attachment detecting unit 109, the terminal for measuring chip 110, the voltage applying unit 111, the electric signal measuring unit 112, the terminal for storing medium 113, the determination program storing unit 114, the instruction content storing unit 115, the stage determination button 116, and a CPU 1006.

Here, the numerical marks assigned to the function blocks in the drawing represent units having the same function as the function blocks in FIG. 4 have, and each of the numerical marks describes an identical function to that in FIG. 4. Thus, the descriptions of the functional blocks having the same numerical numbers shall be omitted.

The stage determination button 116 activates a stage determination program.

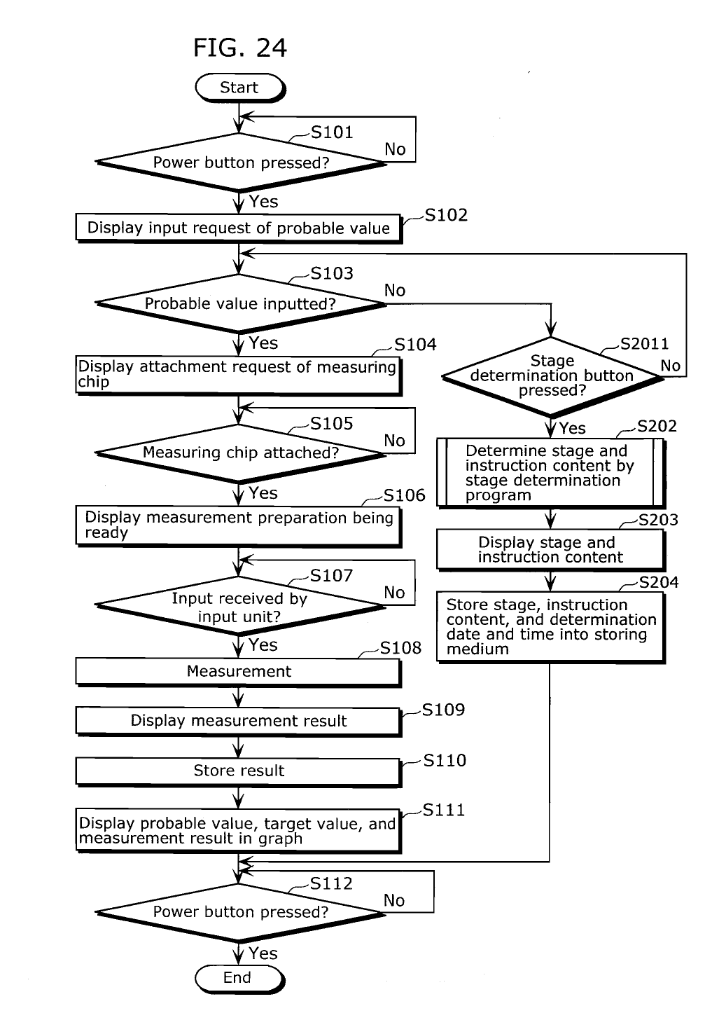

The CPU 1006 is a processing unit for executing various kinds of processing. Details of the processing which the CPU 1006 executes shall be described, referring to FIG. 24.

Next, processing which the lifestyle habit improvement supporting apparatus 10 in the second embodiment executes shall be described, referring to FIGS. 9, 15, and 24.

FIG. 24 is a flowchart showing the processing which the lifestyle habit improvement supporting apparatus 10 in the second embodiment executes.

The CPU 1006: executes, as shown in FIG. 9, the same processing as: the judgment of the power button 101 being pressed (S101 in FIG. 9), and the display processing of the input request of a probable value (S102 in FIG. 9) (S101 and S102); and then judges whether or not the probable value has been inputted (S103). When judging that the probable value has been inputted (S103: Yes), the CPU 1006 executes, as shown in FIG. 9, processing from the display processing of the attachment request of the measuring chip 3 (S104 in FIG. 9) to the display processing of the graph showing the result (S104 in FIG. 9) (S104 through S111).

When judging that the probable value has not been inputted (S103: No), the CPU 1006 judges whether or not the stage determination button 116 has been pressed (S2011). When judging that the stage determination button 116 has not been pressed (S2011: No) the CPU 1006 returns to the input judgment processing of the probable value (S103).

When judging that the probable value has been inputted (S103: Yes), the CPU 1006 executes, as shown in FIG. 15, from the determination processing of a stage and an instruction content (S204 in FIG. 15) to the storing processing into the storing medium 2 (S206 in FIG. 15) (S202 through S206).

Then, the CPU 1006 judges whether or not the power button 107 has been pressed (S112). When judging that the power button 107 has not to be pressed (S112: No), the CPU 1006 stands by until the power button is pressed. When judging that the power button 107 has been pressed (S112: No), the CPU 1006 cuts the electricity and finishes the processing.

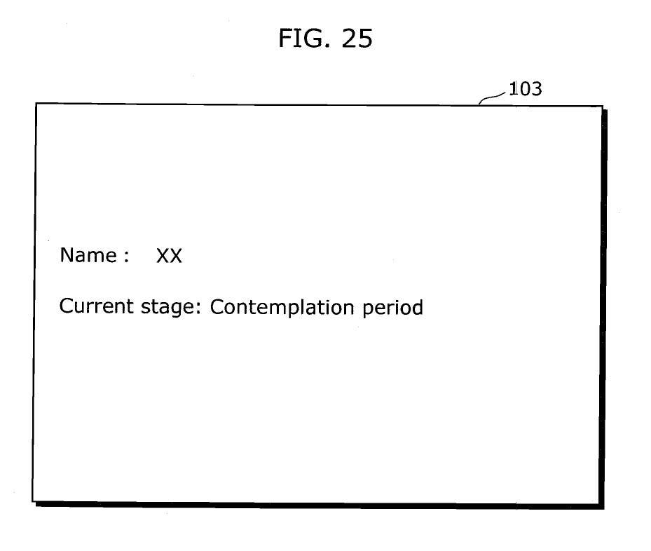

FIG. 25 is a diagram showing a display example, of a stage at which the subject is, displayed on the displaying unit 103 of the lifestyle habit improvement supporting apparatus 10 in the second embodiment. The stage at which the subject is displayed by the subject and a doctor performing an input on the input unit 102 to cause the stage at which the subject is to be displayed.

The displaying unit 103 displays the “name” and the “current stage”. The drawing exemplifies the case where the name is “XX”, and the current stage is the “contemplation stage”.

Further, the stage, and an instruction and advice may be associated and then be displayed simultaneously. When the lifestyle habit improvement supporting apparatus 10 judges the stage to be the “second preparation stage”, the displaying unit 103 presents a low-salt menu along with the current stage.

Use of the above described stage determining program enables the subject's state (stage) to be evaluated from objective data, so that the doctor can comprehend the stage accurately and provide an appropriate treatment to the subject. The subject can also check the stage as needed. Thus, the use of the program effectively motivates the subject to improve his or her lifestyle.

It is noted that, in the embodiment, the input scheme of the target value and the target number of times is the same as that of the first embodiment. A lifestyle habit improvement instructor uses a computer terminal to store the target value and the target number of times into a storing medium. This input scheme, however, is not limited to the above; instead, the target value and the target number of times may be inputted on the input unit 102 in the lifestyle habit improvement supporting apparatus 10.

The lifestyle habit improvement supporting apparatus in the first and the second embodiments of the present invention has been described above; however, the present invention shall not be limited to these embodiments.

For example, in the first embodiment, the cycle, which the subject working on the stage at which the subject is, is set to one month, and the cycle which the subject receives an instruction for the lifestyle habit improvement is set to one month. In the second embodiment, the cycle which the subject receives an instruction for the lifestyle habit improvement is set to one month. These cycles are, however, not limited to one month. Generally, these periods are decided by the instructor for lifestyle habit improvement determining an appropriate cycle for the subject.

In the embodiments of the present invention, salinity concentration in urine is utilized as information representing a lifestyle habit on the subject's salt intake; instead, a value, into which salt intake a day is converted, out of a value of the salinity concentration in urine may also be utilized. For example, by correcting salinity concentration in urine in a single urination with either creatine concentration or voided volume, urinary salt excretion a day may be calculated to be utilized. In that case, salt intake a day, not salinity concentration, is inputted and stored as the target value, the probable value, and the measurement value shown in FIG. 6. This makes a unit of marking easier for the subject to comprehend (gram: g), which provides an instruction having a greater effect.

In addition, other than the measurement value of salinity concentration in urine exemplified in the first and the second embodiments, any given information showing the subject's lifestyle habit may be utilized as far as the information is a measurement value indicating the subject's lifestyle habit. As the information, for example, biological information, such as such as urine and concentration of a substance in blood, and exercise ha it such as the number of steps taken a day, are pointed out.

Further, as an example in the embodiments, a notifying scheme of a result; that is for example a stage and an instruction content and advice by the lifestyle habit improvement apparatus, is to display on the displaying unit. However, the scheme is not limited to this. For example, the scheme may utilize speech or a printout on paper to notify. The scheme to provide an audio notification is effective when the subject is visually impaired and has difficulty in reading the display. The scheme to provide notification on a printout on paper enables the subject to check his or her state anytime since the result remains in the subject's hand, and can provide effective support for improving the subject's lifestyle. In the above cases, the lifestyle habit improvement supporting apparatus includes either a microphone or an audio circuit for providing the audio notification, or a printing unit for printing.

The lifestyle habit improvement supporting apparatus may also be configured to include these notifying schemes, that is two or more functions out of displaying, audio, and printing. In the above case, the lifestyle habit improvement supporting apparatus includes one of a displaying unit, a microphone and an audio circuit, and a printing unit.

Moreover, in the embodiments, the lifestyle habit improvement supporting apparatus may also be configured to establish a connection to another PC, PDA, and cellular phone via the public circuit network, a LAN (Local Area Network), and a WAN (Wide Area Network). This allows the input and output data to be given and received between the lifestyle habit improvement supporting apparatus and the other PC, PDA, and cellular phone. Specifically, output data of the lifestyle habit improvement supporting apparatus; namely the details, such as a stage, and an instruction content and advice, for example, is transmitted to the subject's cellular phone. This allows the subject to check result data anytime, and provides the subject an effective support for his or her lifestyle improvement. In this case, the public circuit network, the LAN, and the WAN may be wired or wireless.

### INDUSTRIAL APPLICABILITY

The present invention can be applicable to apparatuses for supporting a person in need of lifestyle habit improvement. More particularly, the present invention is applicable to a lifestyle habit improvement supporting apparatus and a method there of for presenting an appropriate and effective instruction and an instruction method to a person subject to a lifestyle-related disease.

1. A lifestyle habit improvement supporting apparatus which supports lifestyle habit improvement of a subject, said apparatus comprising:
a measurement value obtaining unit operable to obtain a measurement value reflecting a lifestyle habit of the subject; and
a stage judging unit operable to judge a stage at which the subject working on lifestyle habit improvement is, based on either the measurement value obtained by said measurement value obtaining unit, or the number of measurements representing the number of pieces of data of the measurement value obtained by said measurement value obtaining unit.

2. The lifestyle habit improvement supporting apparatus according to claim 1, further comprising
a target number of measurements obtaining unit operable to obtain the target number of measurements showing the target number of times for which the subject measures the measurement value,
wherein said stage judging unit is operable to judge the stage at which the subject working on lifestyle habit improvement is, based on the number of measurements, and the target number of measurements.

3. The lifestyle habit improvement supporting apparatus according to claim 1, further comprising
a target measurement value obtaining unit operable to obtain a target measurement value showing a target of the measurement value of the subject,
wherein said stage judging unit is operable to judge the stage at which the subject working on lifestyle habit improvement is, based on the measurement value, the number of measurements, and the target measurement value.

4. The lifestyle habit improvement supporting apparatus according to claim 1,
wherein said measurement value obtaining unit is operable to obtain the measurement value corresponding to a measurement date within a certain period, and
said stage judging unit is operable to judge the stage at which the subject working on lifestyle habit improvement is, based on either the measurement value or the number of measurements corresponding to the measurement date within the certain period.

5. The lifestyle habit improvement supporting apparatus according to claim 1,
wherein said measurement value obtaining unit is operable to obtain the measurement value and a probable value which is a measurement value that the subject assumes, and
said stage judging unit is operable to judge the stage at which the subject working on lifestyle habit improvement is based on the measurement value, the probable value, and the number of measurements.

6. The lifestyle habit improvement supporting apparatus according to claim 5,
wherein said measurement value obtaining unit is operable to obtain the measurement value and the probable value which correspond to a measurement date within a certain period, and
said stage judging unit is operable to judge the stage at which the subject working on lifestyle habit improvement is based on the measurement value, the number of measurements, and the probable value which correspond to the measurement date within the certain period.

7. The lifestyle habit improvement supporting apparatus according to claim 1, further comprising
an instruction deciding unit operable to decide an instruction method and advice suitable for the stage at which the subject working on lifestyle habit improvement is, by referring, in accordance with a result of the judgment by said stage judging unit, to a table storing stages of working on lifestyle habit improvement, and instruction methods and advice, suitable for the stage of working for lifestyle habit improvement is.

8. The lifestyle habit improvement supporting apparatus according to claim 1,
wherein said stage judging unit includes a first stage judging sub-unit operable to compare the number of measurements and a first threshold value, and, based on a result of the comparison, to judge the stage at which the subject working on lifestyle habit improvement is as a first stage.

9. The lifestyle habit improvement supporting apparatus according to claim 8,
wherein said first stage judging sub-unit is operable to compare the number of measurements and the first threshold value, and, in the case where the number of measurements is below the first threshold value, to judge the stage at which the subject working on lifestyle habit improvement is as a precontemplation stage in which the subject is uninterested in lifestyle habit improvement.

10. The lifestyle habit improvement supporting apparatus according to claim 2,
wherein said stage judging unit includes a second stage judging sub-unit operable to compare the number obtained by multiplying the target number of measurements by a second threshold value and the number of measurements, and, based on a result of the comparison, to judge the stage at which the subject working on lifestyle habit improvement is as a second stage.

11. The lifestyle habit improvement supporting apparatus according to claim 10,
wherein said second stage judging sub-unit is operable to compare the number obtained by multiplying the target number of measurements by the second threshold value and the number of measurements, and, in the case where the number is equal to or less than the number of measurements, to judge the stage at which the subject working on lifestyle habit improvement is as a contemplation stage in which the subject starts to be interested in lifestyle habit improvement.

12. The lifestyle habit improvement supporting apparatus according to claim 5,
wherein said stage judging unit includes a third stage judging sub-unit operable to compare a ratio of the number of pieces of data to the number of measurements and a third threshold value, and, to judge, based on a result of the comparison, the stage at which the subject working on lifestyle habit improvement is as a third stage, the data having a discrepancy degree between the measurement value and the probable value to be equal to or less than a certain value.

13. The lifestyle habit improvement supporting apparatus according to claim 12,
wherein said third stage judging sub-unit is operable to compare the ratio of the number of pieces of data to the number of measurements and the third threshold value, and, in the case where the ratio is smaller than the third threshold value, to judge the stage at which the subject working on lifestyle habit improvement is as a first preparation stage in which the subject start, working on lifestyle habit improvement but has not acquired correct knowledge yet, the data having the discrepancy degree between the measurement value and the probable value to be equal to or less than a certain value.

14. The lifestyle habit improvement supporting apparatus according to claim 3,
wherein said stage judging unit includes a fourth stage judging sub-unit operable to compare ratio of the number of pieces of data to the number of measurements and a fourth threshold value, and, based on a result of the comparison, to judge the stage, at which the subject working on lifestyle habit improvement is, to be a fourth stage, the data indicating that the measurement value is equal to or less than the target measurement value.

15. The lifestyle habit improvement supporting apparatus according to claim 14,
wherein said fourth stage judging sub-unit is operable to compare the ratio of the number of pieces of data to the number of measurements and the fourth threshold value, and, in the case where the ratio is smaller than the fourth threshold value, to judge the stage at which the subject working on lifestyle habit improvement is as a second preparation stage in which the subject is short of utilizing acquired correct knowledge, the data indicating that the measurement value is equal to or less than the target measurement value.

16. The lifestyle habit improvement supporting apparatus according to claim 14,
wherein said fourth stage judging sub-unit is operable to compare the ratio of the number of pieces of data to the number of measurements and the fourth threshold value, and, in the case where the ratio is smaller than the fourth threshold value, to judge the stage at which the subject working on lifestyle habit improvement is as a preparation stage in which the subject starts working on lifestyle habit improvement, the data indicating that the measurement value is equal to or less than the target measurement value.

17. The lifestyle habit improvement supporting apparatus according to claim 3,
wherein said stage judging unit includes a fifth stage judging sub-unit operable to compare a length of a period and a fifth threshold value, and, based on a result of the comparison, to judge the stage at which the subject working on lifestyle habit improvement is as either a fifth stage or a sixth stage the length of the period representing a period that the ratio of the number of pieces of data to the number of measurements is equal to or more than the fourth threshold value, and the data indicating that the measurement value is equal to or less than the target measurement value.

18. The lifestyle habit improvement supporting apparatus according to claim 17,
wherein said fifth stage judging sub-unit is operable to compare the length of the period and the fifth threshold value, and, in the case where the length of the period is shorter than the fifth threshold value, to judge the stage at which the subject working on lifestyle habit improvement is as an action stage in which the subject begins working on lifestyle habit improvement, utilizing correct knowledge, the length of the period representing the period that the ratio of the number of pieces of data to the number of measurements is equal to or more than the fourth threshold value, and the data indicating that the measurement value is equal to or less than the target measurement value.

19. The lifestyle habit improvement supporting apparatus according to claim 17,
wherein said fifth stage judging sub-unit is operable to compare the length of the period and the fifth threshold value, and, in the case where the length of the period is equal to or more than the fifth threshold value, to judge the stage at which the subject working on lifestyle habit improvement is as a maintenance stage in which an appropriate lifestyle habit has been established for health of the subject, the length of the period representing the period that the ratio of the number of pieces of data to the number of measurements is equal to or more than the fourth threshold value, and the data indicating that the measurement value is equal to or less than the target measurement value.

20. The lifestyle habit improvement supporting apparatus according to claim 1,
wherein the value reflecting the lifestyle habit of the subject represents either salinity concentration, or an amount of salt in urine of the subject.

21. The lifestyle habit improvement supporting apparatus according to claim 7, further comprising
a first displaying controlling unit operable to cause, to be displayed, at least one of: the result of the judgment by said stage judging unit; or one of the instruction method or the advice decided by said instruction deciding unit.

22. The lifestyle habit improvement supporting apparatus according to claim 1, further comprising:
a measuring unit operable to measure the measurement value;
a target receiving unit operable to receive an input of a target measurement value showing a target of the measurement value of the subject, and the target number of measurements showing the target of the number of times for which the subject measures the measurement value; and
a storing unit which stores a measurement date and time when the subject measures, the target measurement value and the target number of measurements received by said target receiving unit, and the measurement value measured by said measuring unit,
wherein said measurement value obtaining unit is operable to obtain, from said storing unit, the measurement date and time, the target measurement value, the target number of measurements, and the measurement value.

23. The lifestyle habit improvement supporting apparatus according to claim 1, further comprising:
a measuring unit operable to measure the measurement value;
a target receiving unit operable to receive an input of a target measurement value showing a target of the measurement value of the subject, and the target number of measurement showing the target of the number of times for which the subject measures the measurement value;
a probable value receiving unit operable to receive an input of a probable value; and
a storing unit which stores a measurement date and time when the subject measures, the target measurement value and the target number of measurements received by said target receiving unit, the measurement value measured by said measuring unit, and the probable value received by said probable value receiving unit,
wherein said measurement value obtaining unit is operable to obtain, from said storing unit, the measurement date and time, the target measurement value, the target number of measurements, the measurement value, and the probable value.

24. The lifestyle habit improvement supporting apparatus according to claim 1,
wherein said measurement value obtaining unit is operable to obtain the measurement value, outside said lifestyle habit improvement supporting apparatus, from a storing unit which is a dismountable storing medium.

25. A lifestyle habit improvement supporting method for supporting lifestyle habit improvement of a subject, said method comprising:
a measurement value obtaining step of obtaining a measurement value reflecting a lifestyle habit of the subject; and
a stage judging step of judging a stage at which the subject working on lifestyle habit improvement is, based on either the measurement value obtained in said measurement value obtaining step, or the number of measurements calculated from the number of pieces of data of the measurement value obtained in said measurement value obtaining step.

26. A computer-executable program for supporting lifestyle habit improvement of a subject, said program causing a computer to execute:
a measurement value obtaining step of obtaining a measurement value which is a value reflecting a lifestyle habit of the subject; and
a stage judging step of judging a stage at which the subject working on lifestyle habit improvement is, based on either the measurement value obtained in said measurement value obtaining step, or the number of measurements calculated from the number of pieces of data of the measurement value obtained in said measurement value obtaining step.
